(19) United States
(12) Patent Application Publication
(10) Pub. No.: US 2010/0209508 A1
(43) Pub. Date: Aug. 19, 2010

(54) Substituted Phenylphosphates as Mutual Prodrugs of Steroids and ß-Agonists for the Treatment of Title Pulmonary Inflammation and Bronchoconstriction

(75) Inventors: William Baker, Bellevue, WA (US); Marcin Stasiak, Seattle, WA (US); Charles Bruce Girton, Issaquah, WA (US)

Correspondence Address: WOOD, PHILLIPS, KATZ, CLARK & MORTIMER, 500 W. MADISON STREET, SUITE 3800, CHICAGO, IL, 60661, US

(73) Assignee: CORUS PHARMA, INC., Seattle, WA (US)

(21) Appl. No.: 11/922,258

(22) PCT Filed: Jun. 12, 2006
(86) PCT No.: PCT/US2006/022790

§ 371 Date: Dec. 21, 2009

Related U.S. Application Data
(60) Provisional application No. 60/690,545, filed on Jun. 14, 2005.

(51) Int. Cl. A61K 31/58; C07J 71/00; A61K 9/14; A61P 29/00; A61P 11/08; A61P 11/00; A61P 11/06

(52) U.S. Cl. 424/484; 540/63; 514/174; 540/70

(57) ABSTRACT

A mutual prodrug of a corticosteroid and a substituted phenylphosphate (β-agonist derivative) for formulation for delivery by aerosolization to inhibit pulmonary inflammation and bronchoconstriction is described. The mutual prodrug is preferably formulated in a small volume solution (10-500 μL) dissolved in a quarter normal saline having pH between 5.0 and 7.0 for the treatment of respiratory tract inflammation and bronchoconstriction by an aerosol having mass median average diameter predominantly between 1 to 5μ, produced by nebulization or by dry powder inhaler.

### FIELD OF THE INVENTION

The current invention relates to the preparation of novel, mutual prodrugs of corticosteroids and β-agonists for delivery to the lung by aerosolization. In particular, the invention concerns the synthesis, formulation and delivery of substituted phenylphosphate-steroid as mutual steroid-β-agonist prodrugs such, that when delivered to the lung, endogenous enzymes present in the lung tissue and airway degrade the prodrug releasing a corticosteroid and a β-agonist (e.g. salmeterol, albuterol) at the site of administration. The described mutual prodrugs are formulated as either liquids or dry powders and the formulation permits and is suitable for delivery of the prodrugs to the lung endobronchial space of airways in an aerosol having a mass median average diameter predominantly between 1 to 5μ. The formulated and delivered efficacious amount of substituted phenylphosphate prodrugs is sufficient to deliver therapeutic amounts of both steroid and β-agonist for treatment of respiratory tract diseases, specifically pulmonary inflammation and bronchoconstriction associated with mild to severe asthma, as well as bronchitis or chronic obstructive pulmonary disease (COPD).

### BACKGROUND OF THE INVENTION

Asthma is a chronic inflammatory disease of the airways resulting from the infiltration of pro-inflammatory cells, mostly eosinophils and activated T-lymphocytes (Poston, 1992; Walker, 1991) into the bronchial mucosa and submucosa. The secretion of potent chemical mediators, including cytokines, by these proinflammatory cells alters mucosal permeability, mucus production, and causes smooth muscle contraction. All of these factors lead to an increased reactivity of the airways to a wide variety of irritant stimuli (Kaliner, 1988).

Glucocorticoids, which were first introduced as an asthma therapy in 1950 (Carryer, 1950) remain the most potent and consistently effective therapy for this disease, although their mechanism of action is not yet fully understood (Morris, 1985). Available evidence suggests that at least one mechanism by which they exert their potent anti-inflammatory properties is by inhibiting the release and activity of cytokines, which recruit and activate inflammatory cells such as eosinophils (Schleimer, 1990). Ordinarily, eosinophils undergo the phenomenon of apoptosis or programmed cell death, but certain cytokines such as interleukin 5 (IL-5), interleukin-3 (IL-3), and granulocyte-macrophage colony stimulating factor (GM-CSF) increase eosinophil survival from 1 or 2 days to 4 days or longer and cause eosinophil activation (Kita, 1992). Wallen (1991) was the first to show that glucocorticoids potently block the cytokine's ability to enhance eosinophil survival in a concentration-dependent manner.

Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10^{th} edition, 2001). An obvious solution to systemic side effects would be the delivery of steroid drugs directly to the site of inflammation. Thus, inhaled corticosteroids (ICS) were developed to mitigate the severe adverse effects of oral steroids. While ICS are very effective in controlling inflammation in asthma, they too produce unwanted side effects in the mouth and pharynx (candidiasis, sore throat, dysphonia). The side effects associated with oral glucocorticoid and ICS therapy have led to interest in agents, which exhibit similar antiinflammatory effects. A variety of such agents have been tested. For example, preparations of cyclosporin (Szczeklik, 1991; Mungan, 1995), methotrexate (Dyer, 1991), troleandomycin (TAO) (Wald, 1986; Shivaram, 1991), and gold (Szczeklik, 1991; Dykewicz, 2001; Bernstein, 1988) have been used in attempts to wean patients off orally administered steroids. Similarly, leukotriene receptor antagonists (e.g. montelukast [Singulair®] and zafirlukast [Accolate®]) (Korenblat, 2001; Dykewicz, 2001; Wechsler, 1999), colchicine (Fish, 1997), salmeterol (Lazarus, 2001; Lemanske, 2001), and anti-immunoglobulin E (IgE) (Dykewicz, 2001) have been used with limited success in efforts to wean patients off inhaled steroids. However to date, no completely satisfactory substitute for glucocorticoid therapy has been identified.

Bronchodilators such as albuterol or salmeterol relax airway smooth muscles by blocking opposing active contraction. Many of these bronchodilators activate the β₂-adrenoreceptor as their mode of action. The result is the dilation by 2-3 mm in diameter of small peripheral airways, which are the site of action in both asthma and COPD.

In consideration of all problems and disadvantages connected with the adverse side effect profile of ICS (candidiasis, sore throat, dysphonia) and of β-agonists (tachycardia, ventricular dysrhythmias, hypokalemia) it would be highly advantageous to provide a water-soluble, mutual steroid-β-agonist prodrug to mask the pharmacological properties of both steroids and β-agonists until such a prodrug reaches lungs, thereby mitigating the oropharyngeal side effects of ICS and cardiovascular side-effects of β-agonists. Such a mutual steroid-β-agonist prodrug would be effectively delivered to the endobronchial space and converted to active drugs by the action of lung enzymes, thereby delivering to the site of inflammation and bronchoconstriction a therapeutic amount of both drugs.

The mutual steroid-β-agonist prodrug would provide a therapeutic agent to dilate the airway, thereby allowing the second component (steroid) to effectively penetrate and reach the site of inflammation. It would be highly desired to have a mutual prodrug of a β-agonist and a corticosteroid that produces sustained release of both drugs at the site of administration. Additionally, it would be highly desirable to have such a mutual prodrug to be poorly absorbed from the lung and to be sufficiently water soluble allowing the flexibility in its formulation and delivery system.

It is therefore a primary object of this invention to provide novel substituted phenylphospates as mutual prodrugs of a steroid and a β-agonist.

It is a further object of this invention to provide a composition of the mutual prodrugs, which is stable as a liquid or solid dosage form for nebulization or dry powder delivery. Such composition contains sufficient but not excessive concentration of the active substance which can be efficiently aerosolized by metered-dose inhalers, nebulization in jet, ultrasonic, pressurized, or vibrating porous plate nebulizers or by dry powder into aerosol particles predominantly within the 1 to 5μ size range, and which salinity and pH are adjusted to permit generation of a mutual prodrug aerosol well tolerated by patients, and which formulation further has an adequate shelf life.

### SUMMARY OF THE INVENTION

The present invention is directed to substituted phenylphosphates as mutual prodrugs of steroids and β-agonist and their use and formulation for delivery by inhalation as a method to treat pulmonary inflammation and bronchoconstriction. The prodrug incorporates charged phosphate and quaternary ammonium groups, which renders the molecule highly polar and water soluble and imparts its affinity to lung DNA and protein thus minimizing rapid systemic absorption, as well as absorption due to swallowing. Furthermore, since the mutual prodrug cannot be activated in absence of alkaline phosphatase, the oropharyngeal and systemic side effects are eliminated due to the minimal activity of that enzyme in saliva, and low phosphatase activity in plasma, as compared to other tissues, including lungs (Testa and Mayer, 2003).

More specifically, the present invention is directed to a compound of the formula I or II

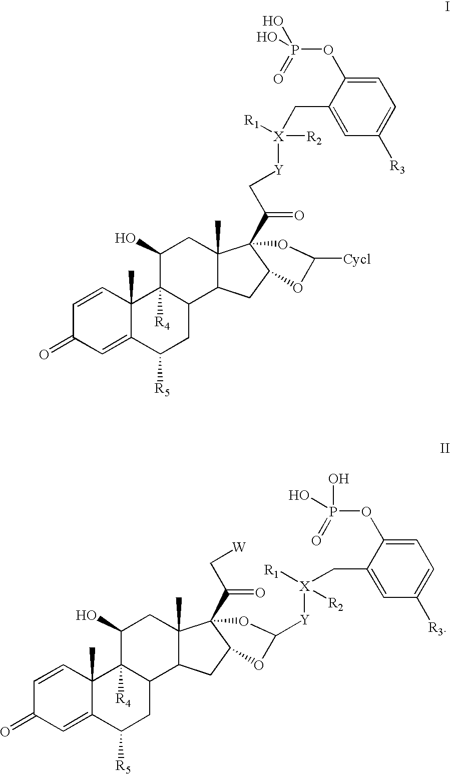

and pharmaceutical acceptable salts thereof, wherein:

X is S, N or a nitrogen-containing heterocycle in which the nitrogen atom in the heterocycle is linked to R₁ and R₂;
W is selected from the group consisting of Cl, F, OH, ONO₂, OCO-alkyl, OCO-aryl, CN, S-alkyl, and S-aryl;
Cycl is cycloalkyl or cycloalkyl with carbon atom(s) substituted with S or O;
Y is either absent or —Z(CH₂)n where n=0-6 and Z is S, O, N or N-alkyl;
R₁ and R₂ are independently selected from the group consisting of hydrogen, aryl, loweralkyl and substituted loweralkyl, or absent, or taken together to form a nonaromatic ring having 2-10 atoms selected from C, O, S, and N;

### R₃ is

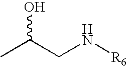

where R₆ is an alkyl group of 1-12 carbon atoms, arylalkyl or substituted arylalkyl with 1-3 CH₂ groups in the carbon chain substituted with atom(s) selected from O, S and N, and
R₄ and R₅ are independently H, Cl or F.
Presently preferred embodiments of this invention include compounds of formula I, wherein:
Cycl is cyclohexyl, R₁ is methyl, R₂ is absent, Y is N(CH₂)ₙ linked with X to form a piperazine ring,

### R₃ is

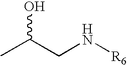

where R₆ is (CH₂)₆O(CH₂)₄Ph or tert-butyl, R₄ is F and R₅ is H.

Other preferred embodiments include compounds of formula I, wherein: Cycl is cyclohexyl, R₁ is methyl, R₂ is absent, Y is absent, X is S,

### R₃ is

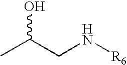

where R₆ is (CH₂)₆O(CH₂)₄Ph or tert-butyl, R₄ is F and R₅ is H.

Other preferred embodiments of this invention include compounds of formula II wherein: Y, R₁ and R₂ are absent and X forms a 4-tetrathiohydropyranyl ring, W is OH or CN

### R₃ is

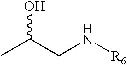

where R₆ is (CH₂)₆O(CH₂)₄Ph or tert-butyl, R₄ is F and R₅ is H.

Other preferred embodiments of this invention include compounds of formula II wherein: Y, R₁ and R₂ are absent and X forms a 3-pyridyl ring, W is OH or CN

### R₃ is

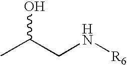

where R₆ is (CH₂)₆O(CH₂)₄Ph or tert-butyl, R₄ is F and R₅ is H.

Examples of presently preferred compounds of this invention include:
Salmeterol-phosphate-16,17-[(Cyclohexylmethylene)bis(oxy)]-9-fluoro-11-hydroxy-21-(4-methylpiperazinium)-pregna-1,4-diene-3,20-dione[11β,16α(R)] (Example 107);
Albuterol-phosphate-16,17-[(Cyclohexylmethylene)bis(oxy)]-9-fluoro-11-hydroxy-21-(4-methylpiperazinium)-pregna-1,4-diene-3,20-dione[11β,16αα(R)] (Example 109);
Salmeterol-phosphate-16,17-[(Cyclohexylmethylene)bis(oxy)]-9-fluoro-11-hydroxy-21-methylsulfonium-pregna-1,4-diene-3,20-dione[11β,16α(R)] (Example 115);
Albuterol-phosphate-16,17-[(Cyclohexylmethylene)bis(oxy)]-9-fluoro-11-hydroxy-21-methylsulfonium-pregna-1,4-diene-3,20-dione[11β,16α(R)] (Example 117);
Salmeterol-phosphate-16,17-[(Tetrahydro-thiopyranylium)bis(oxy)]-9-fluoro-11,21-dihydroxy-pregna-1,4-diene-3,20-dione[11β,16α(R)] (Example 120);
Albuterol-phosphate-16,17-[(Tetrahydro-thiopyranylium)bis(oxy)]-9-fluoro-11,21-dihydroxy-pregna-1,4-diene-3,20-dione[11β,16α(R)] (Example 122);
Salmeterol-phosphate-16,17-[Pyridynium-3-methylene)bis(oxy)]-9-fluoro-11,21-dihydroxypregna-1,4-diene-3,20-dione[11β,16α] (Example 133);
Albuterol-phosphate-16,17-[Pyridynium-3-methylene)bis(oxy)]-9-fluoro-11,21-dihydroxypregna-1,4-diene-3,20-dione[11β,16α] (Example 135);
Salmeterol-phosphate-16,17-[Pyridynium-3-methylene)bis(oxy)]-9-fluoro-11-hydroxy-21-cyano-pregna-1,4-diene-3,20-dione[11β,16α] (Example 137); and
Albuterol-phosphate-16,17-[Pyridynium-3-methylene)bis(oxy)]-9-fluoro-11-hydroxy-21-cyano-pregna-1,4-diene-3,20-dione[11β,16α] (Example 139).

The present invention also relates to the process of synthesis of the preferred mutual prodrugs listed above, as well as to novel steroids released by the action of lung enzymes (specifically alkaline phosphatase) from the preferred mutual prodrugs of this invention.

The novel steroids are described by formula III,

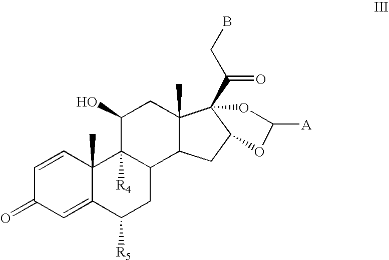

or pharmaceutically acceptable salts thereof, wherein:

A is cycloalkyl (with carbon atom(s) optionally substituted with S, O or NR₁), pyridyl or substituted pyridyl;
B is selected from the group consisting of NR₁R₂, imidazolyl, CN, SCN, SR₁, Cl, F, OH, ONO₂, OCO-alkyl and OCO-aryl;
R₁ and R₂ are independently selected from the group consisting of hydrogen, aryl, heteroaryl, loweralkyl and substituted loweralkyl, or absent, or taken together to form a nonaromatic ring having 2-10 atoms selected from C, O, S, and N.

Presently preferred novel steroids of this invention of formula III include:
16,17-[(Cyclohexylmethylene)bis(oxy)]-9-fluoro-11-hydroxy-21-(4-methylpiperazin-yl)-pregna-1,4-diene-3,20-dione[11β,16α(R)] (Example 27);
16,17-[(Cyclohexylmethylene)bis(oxy)]-9-fluoro-11-hydroxy-21-methylthio-pregna-1,4-diene-3,20-dione[11β,16α(R)] (Example 51);
16,17-[(Tetrahydro-thiopyran-4-yl)bis(oxy)]-9-fluoro-11,21-dihydroxy-pregna-1,4-diene-3,20-dione[11β,16α(R)] (Example 53);
16,17-[Pyridynyl-3-methylene)bis(oxy)]-9-fluoro-11,21-dihydroxypregna-1,4-diene-3,20-dione[11β,16α] (Example 62); and
16,17-[Pyridynyl-3-methylene)bis(oxy)]-9-fluoro-11-hydroxy-21-cyano-pregna-1,4-diene-3,20-dione[11β,16α] (Example 83).

The invention also relates to a pharmaceutically acceptable composition for the treatment of a disorder selected from severe to mild asthma, bronchitis, COPD or other diseases related to pulmonary inflammation and bronchoconstriction, which comprises a therapeutically effective amount, preferably from about 10 μg to about 1000 μg, of at least one compound of formula I or II or a pharmaceutically acceptable salt thereof, and a pharmaceutically accepted carrier. The composition is preferably administered as an aerosol, most preferably by a dry powder inhaler. The invention also relates to methods of treating such diseases with therapeutically effective amounts of at least one compound of formula I or II or a pharmaceutically acceptable salt thereof.

The invention also relates to a liquid or dry powder formulation of the corticosteroid-β-agonist prodrug combination for the treatment of a disorder selected from severe to mild asthma, bronchitis, and COPD or other diseases related to pulmonary inflammation and bronchoconstriction, which comprises a therapeutically effective amount, preferably from about 10 μg to about 1000 μg, of at least one compound of formula I or II or a pharmaceutically acceptable salt thereof. The composition is preferably administered as an aerosol, most preferably by a dry powder inhaler.

The invention further relates to a method for the prevention and treatment of pulmonary inflammation and bronchoconstriction, comprising administering to a patient in need of such treatment an effective amount of an aerosol formulation comprising about 10 μg to about 1000 μg of the mutual prodrugs of the present invention. Preferably, when the prodrug is delivered to the lung, the phosphate group is cleaved by an endogenous enzyme alkaline phosphatase and the steroid and the β-agonist are individually released in a simultaneous manner.

### BRIEF DESCRIPTION OF THE DRAWINGS

FIG. 1 and FIG. 2 plot the concentration of mutual prodrug and active drugs versus time during enzymatic conversion of the prodrug.

### DETAILED DESCRIPTION OF THE INVENTION

As used herein “aryl” is defined as an aromatic ring substituted with 1-3 groups selected from hydrogen, amino, hydroxy, halo, O-alkyl and NH-alkyl. Aryl can be one or two rings either fused to form a bicylic aromatic ring system or linear as in biphenyl. The aryl group can be substituted with N, S, or O in the ring to produce a heterocyclic system.

The term “alkyl” as used herein refers to a branched or straight chain comprising one to twenty carbon atoms which can optionally comprise one or more atoms selected from O, S, or N. Representative alkyl groups include methyl, butyl, hexyl, and the like.

As used herein “lower alkyl” includes both substituted or unsubstituted straight or branched chain alkyl groups having from 1 to 10 carbon atoms. Representative loweralkyl groups include for example, methyl, ethyl, propyl, isopropyl, n-butyl, tert-butyl, and the like. Representative of halo-substituted, amino-substituted and hydroxy-substituted, lower-alkyl include chloromethyl, chloroethyl, hydroxyethyl, aminoethyl, etc.

As used herein “cycloalkyl” includes a non-aromatic ring composed of 3-10 carbon atoms.
As used herein, the term “halogen” refers to chloro, bromo, fluoro and iodo groups.

The term “substituted heterocycle” or “heterocyclic group” or “heterocycle” as used herein refers to any 3- or 4-membered ring containing a heteroatom selected from nitrogen, oxygen, and sulfur or a 5- or 6-membered ring containing from one to three heteroatoms selected from the group consisting of nitrogen, oxygen, or sulfur; wherein the 5-membered ring has 0-2 double bounds and the 6-membered ring has 0-3 double bounds; wherein the nitrogen and sulfur atom may be optionally oxidized; wherein the nitrogen and sulfur heteroatoms may be optionally quarternized; and including any bicyclic group in which any of the above heterocyclic rings is fused to a benzene ring or another 5- or 6-membered heterocyclic ring independently defined above. Heterocyclics in which nitrogen is the heteroatom are preferred. Fully saturated heterocyclics are also preferred. Preferred heterocycles include: diazapinyl, pyrryl, pyrrolinyl, pyrrolidinyl, pyrazolyl, pyrazolinyl, pyrazolidinyl, imidazoyl, imidazolinyl, imidazolidinyl, pyridyl, piperidinyl, pyrazinyl, piperazinyl, azetidinyl, pyrimidinyl, pyridazinyl, oxazolyl, oxazolidinyl, isoxazolyl, isoazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, triazolyl and benzothienyl groups.

Heterocyclics can be unsubstituted or monosubstituted or disubstituted with substituents independently selected from hydroxy, halo, oxo (C═O), alkylimino (RN═, wherein R is a lower alkyl or alkoxy group), amino, alkylamino, dialkylamino, acylaminoalkyl, alkoxy, thioalkoxy, loweralkyl, cycloalkyl or haloalkyl. The most preferred heterocyclics include imidazolyl, pyridyl, piperazinyl, azetidinyl, thiazolyl, triazolyl, benzimidazolyl, benzothiazolyl and benzoxazolyl.

As used herein, the term “pharmaceutically acceptable salts” refers to the salt with a nontoxic acid or alkaline earth metal salts of the compounds of formula I or II. These salts can be prepared in situ during the final isolation and purification of the compounds of formula I or II, or separately, by reacting the base or acid functions with a suitable organic or inorganic acid or base, respectively. Representative acid salts include hydrochloride, hydrobromide, bisulfate, acetate, oxalate, valerate, oleate, palmitate, stearate, laurate, borate, benzoate, lactate, citrate, maleate, tartrate salts, and the like. Representative alkali metals of alkaline earth metal salts include sodium, potassium, calcium, and magnesium.

As used herein, the term “alkoxy” refers to —O—R wherein R is lower alkyl as defined above. Representative examples of lower alkoxy groups include methoxy, ethoxy, tert-butoxy, and the like.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “normal saline” means water solution containing 0.9% (w/v) NaCl.

The term “diluted saline” means normal saline containing 0.9% (w/v) NaCl diluted into its lesser strength.

The term “quarter normal saline” or “¼ NS” means normal saline diluted to its quarter strength containing 0.225% (w/v) NaCl.

The term “prodrug” as used herein refers to a compound in which specific bond(s) of the compound are broken or cleaved by the action of an enzyme or by biological process thereby producing or releasing a drug and compound fragment which is substantially biologically inactive.

The term “mutual prodrug” as used herein refers to a bipartite or tripartite prodrug in which specific bond(s) of the compound are broken or cleaved by the action of an enzyme or by biological process thereby producing or releasing a drug and the carrier which is a synergistic drug of the drug to which it is linked.

The compounds of the invention may comprise asymmetrically substituted carbon atoms. Such asymmetrically substituted carbon atoms can result in the compounds of the invention comprising mixtures of stereoisomers at a particular asymmetrically substituted carbon atom or a single stereoisomer. As a result, racemic mixtures, mixtures of diastereomers, as well as single diastereomers of the compounds of the invention are included in the present invention. The terms “S” and “R” configuration, as used herein, are as defined by the IUPAC 1974 RECOMMENDATIONS FOR SECTION E, FUNDAMENTAL STEREOCHEMISTRY, Pure Appl. Chem. 45:13-30 (1976). The terms α and β are employed for ring positions of cyclic compounds. The α-side of the reference plane is that side on which the preferred substituent lies at the lower numbered position. Those substituents lying on the opposite side of the reference plane are assigned β descriptor. It should be noted that this usage differs from that for cyclic stereoparents, in which “α” means “below the plane” and denotes absolute configuration. The terms α and β configuration, as used herein, are as defined by the CHEMICAL ABSTRACTS INDEX GUIDE-APPENDIX IV (1987) paragraph 203.

The present invention also relates to the processes for preparing the compounds of the invention and to the synthetic intermediates useful in such processes, as described in detail below.

### I. Preparation of the Compounds of the Invention

The compounds of the present invention can be prepared by the processes illustrated in Schemes I-VII.

A convergent route to a mutual corticosteroid-β-agonist prodrug involves:

a) synthesis of the activated phosphate-β-agonist derivatives (Scheme I, II and III);
b) preparation of the steroid analogs (Schemes IV and V);
c) alkylation of the steroid analogs with the activated β-agonist derivative, followed by the final deprotection (Schemes VI and VII).

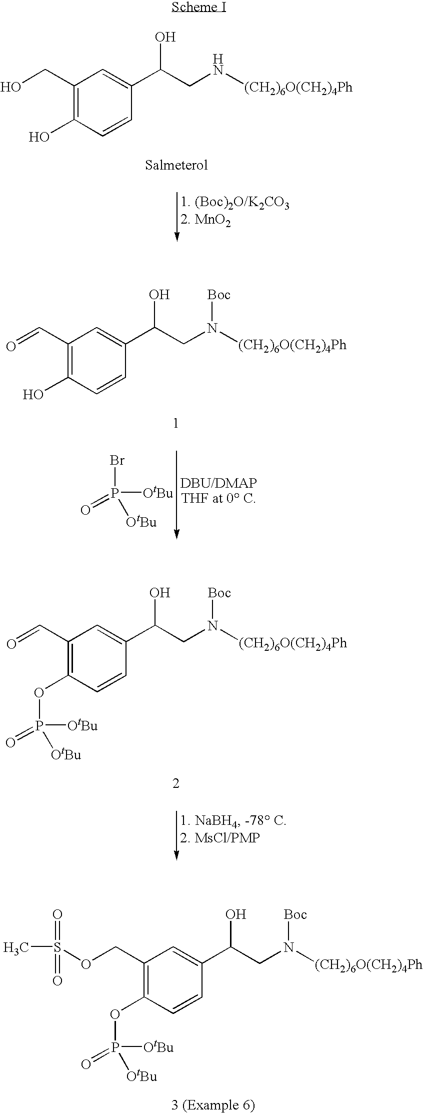

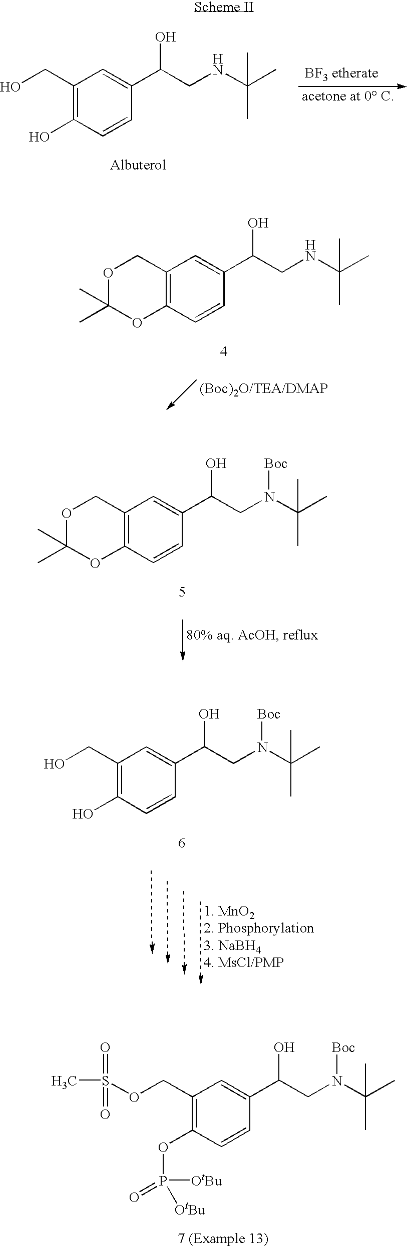

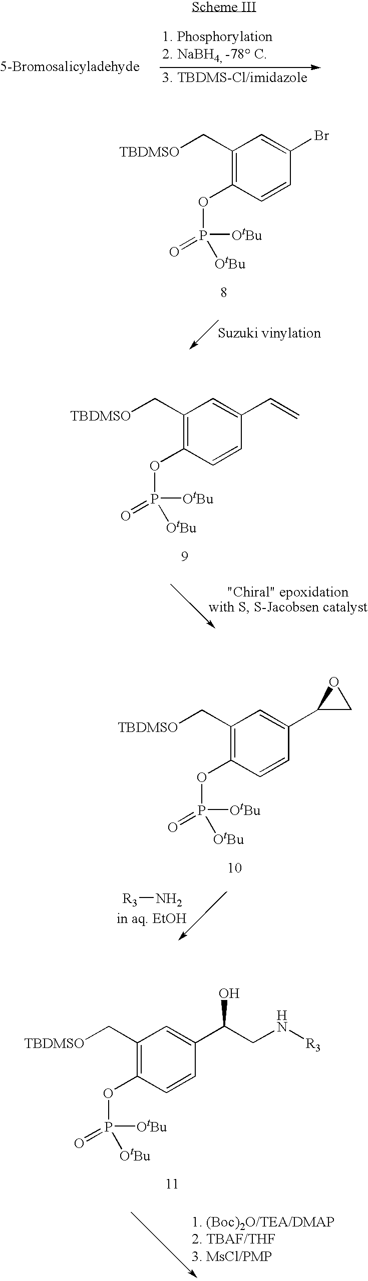

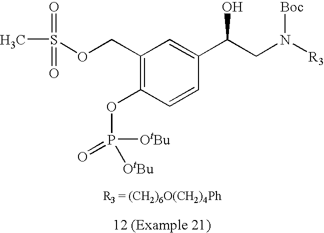

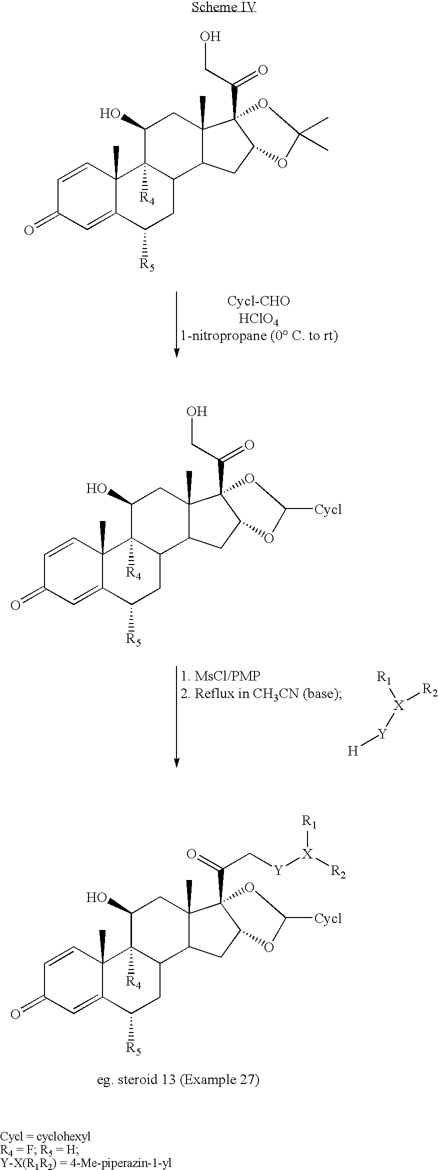

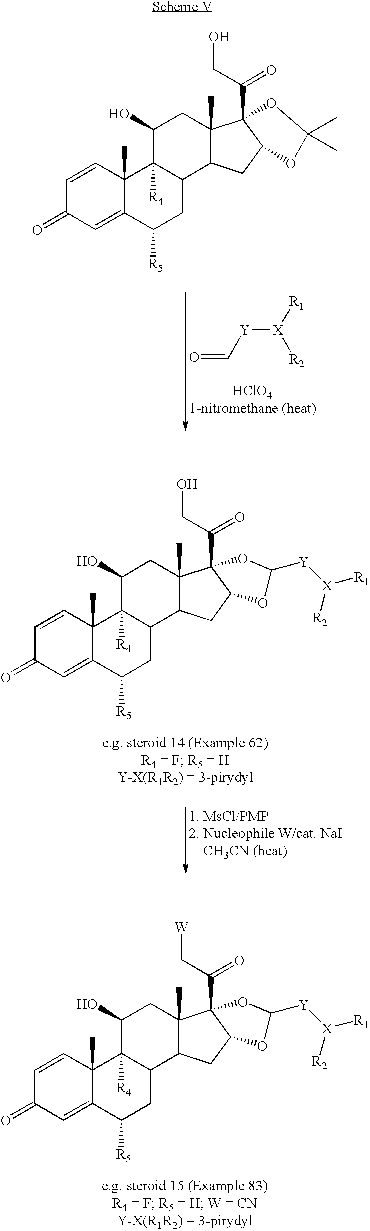

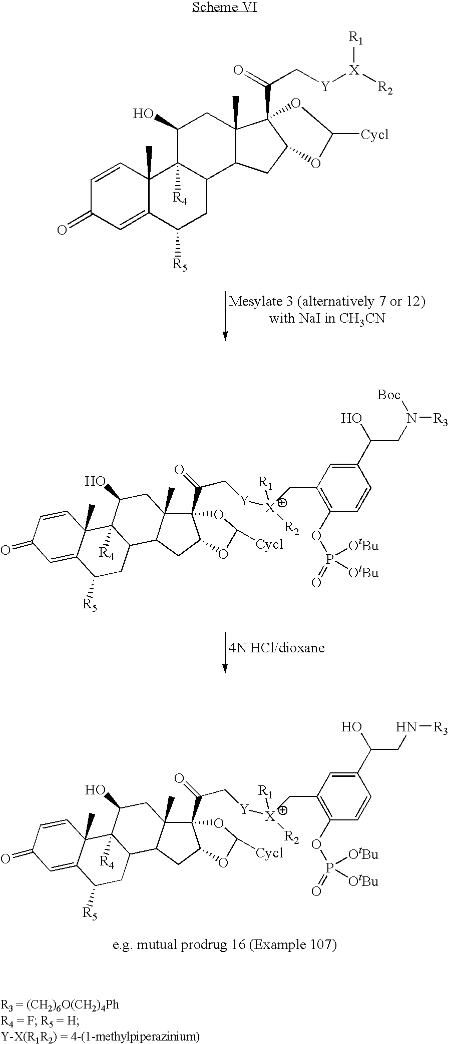

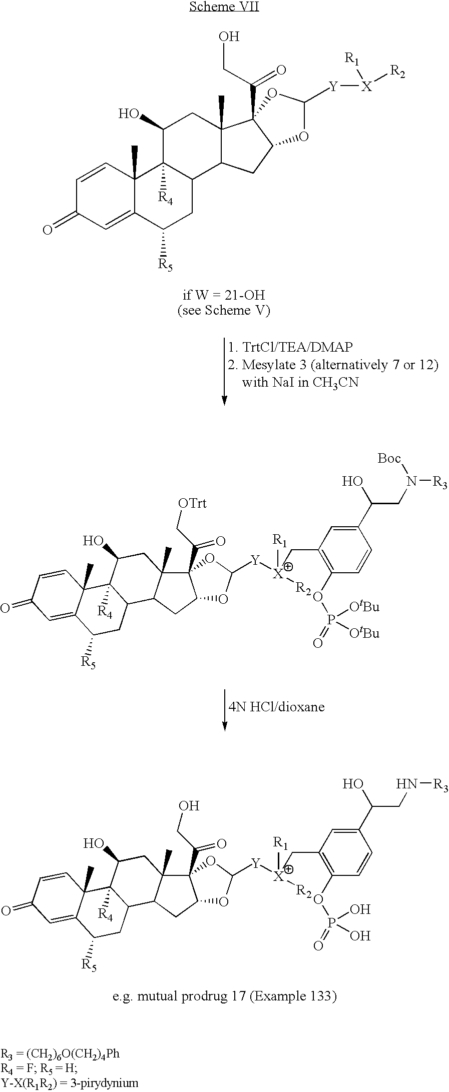

Synthesis of the phosphate-functionalized protected β-agonist derivative is shown in Schemes I-III. Commercially available racemic salmeterol (or prepared according to Rong and Ruoho, 1999) was protected with t-butoxycarbonyl, followed by the selective oxidation of the primary, benzylic alcohol to aldehyde with activated MnO₂, yielding compound 1 (Example 3). In this manner the primary alcohol is protected in a latent fashion, and the acidity of the phenolic moiety is increased helping the selectivity of the subsequent phosphorylation. Consequently the reaction with a slight excess of phosphobromidate (prepared as described in Example 1) proceeded cleanly, yielding the phosphate 2 in good yield and purity (Example 4). The reduction of the aldehyde moiety with sodium borohydride carried out at low temperature (−78° C. to 0° C.) produced the diol, which was selectively sulfonylated using methanesulfonyl chloride (MsCl) in the presence of 1,2,2,6,6-pentamethylpiperidine (PMP) to give the primary mesylate 3 (Example 6) used in the alkylation linking of the steroid and β-agonist into a mutual prodrug.

In the case when a bulky, sterically hindered R₃ substituent is present in the β-agonist moiety (e.g. when R₃ equals tert-butyl for albuterol), additional protective group manipulation is necessary prior to the phosphorylation, as illustrated in Scheme II.

Commercially available racemic albuterol (salbutamol) was temporarily protected in the form of O,O-isopropylidene (Stevens, 1999), therefore enabling selective protection of the secondary, sterically hindered amine by prolonged (48 hours) treatment with excess di-tert-butyl dicarbonate, yielding the derivative 5 (Example 8). The removal of the isopropylidene protection was accomplished by brief heating in the refluxing 80% (v/v) aqueous acetic acid, during which the Boc moiety stays intact (Example 9). Thus obtained N-Boc-albuterol (6) was transformed into the phosphorylated derivative 7 through a four-step synthetic sequence identical to one described in Scheme I (Examples 10-13).

The synthetic process towards the optically pure, phosphorylated β-agonist derivative is illustrated on Scheme III. 5-Bromosalicylaldehyde was phosphorylated and the aldehyde moiety reduced as described in the earlier paragraph, and the thus formed alcohol moiety can be protected by treatment with tert-butyldimethylsilyl chloride in the presence of imidazole, yielding the compound 8 (Examples 13-15). The presence of a bromine atom allows the C—C bond formation in the following step. The trivinylboroxine-pyridine complex in the presence of catalytic amounts of tricyclohexylphosphine and palladium (II) acetate was used to introduce the vinyl substituent using the Suzki method (Example 17). Thus formed compound 9 undergoes asymmetric hypochlorite-NMMO oxidation in the presence of a catalytic amount of (S,S)-(+)N,N′-bis(3,5-di-tert-butylsalicylidene)-1,2-cyclohexanediaminomanganese (III) chloride (Jacobsen, 1991) yielding the S-epoxide 10 with enantiomeric purity exceeding 90%. If desired, the R,R-version of the Jacobsen's catalyst can be used to prepare the optical antipode of 10. The epoxide opening was accomplished by the nucleophilic attack with the amine bearing the R₃ moiety. On the route to the chiral salmeterol derivative, the 6-(4-phenylbutoxy)-hexylamine (Example 16) was reacted with compound 10 in 95% aqueous ethanol at slightly elevated temperature (see Example 19). The secondary amine 11 thus formed was protected by treatment with di-t-butyl dicarbonate in the presence of triethylamine and catalytic DMAP in anhydrous THF. The silyl group was then removed using tetrabutylammonium fluoride and the resulting diol was selectively mesylated, as described in previous paragraphs, to give the optically pure R-mesylate 12 in good yield (Example 21).

Scheme IV describes the synthesis of prednisolone derivatives modified with the 16,17-cycloalkylidene moiety and with the 21-substituent allowing the linkage of the β-agonist moiety through the quaternizable nitrogen atom, or alternatively via a sulfonium salt. Using the modification of the procedures described by Gutterer (1994 and 2002) the 16-α-hydroxyprednisolone derivatives (e.g. desonide or triamcinolone acetonide) were reacted at 0° C. to room temperature with selected cycloalkyl carboxaldehydes. In certain cases (e.g. cyclohexyl) the 22-R diastereoisomer (confirmed by the 2D NMR methods) was obtained as the major epimer with diastereoisomeric purity exceeding 90% (Example 22 and 23). Further modification of the steroid analogs was accomplished by the selective activation of the 21-hydroxyl group through the intermediate sulfonate esters, advantageously methanesulfonates (see Examples 24 and 25). The mesylate was displaced by the nucleophilic substitution (Examples 26-51) with the amine, thiol or a heterocycle by heating in the refluxing acetonitrile in presence of a base (e.g. anhydrous powdered potassium carbonate). Compounds described in Examples 52-55 illustrate the case when the 16,17-cycloalkylidene moiety introduced via transacetalization contains the sulfur atom serving as a handle for linking the phosphorylated β-agonist moiety (see mutual prodrugs described in Examples 120 and 122).

Scheme V describes the synthesis of prednisolone derivatives modified with the 16,17-acetal moiety derived from the heterocyclic aldehydes containing nitrogen atom capable of linking the β-agonist moiety through the quaternary ammonium salt. In case of those less reactive aldehydes the acetal formation (Examples 56-81) required in most cases heating (80° C.) and increased amount of perchloric acid (4 equivalents) as compared to conditions applied for cycloalkyl aldehydes. Also the use of the more polar solvent 1-nitromethane (instead of 1-nitropropane) for transacetalization proved to be advantageous ensuring the homogeneity of the mixture throughout the reaction. Further modification of the 16,17-acetals was carried out similarly as described in Scheme IV via intermediate mesylates synthesized by the usual procedure (MSCl in presence of PMP in dichloromethane). Final substitution was accomplished by heating the respective mesylates with a nucleophilic reagent (e.g. cyanide for Examples 82-103) in the presence of a catalytic amount of sodium iodide.

Schemes VI and VII illustrate the final assembly of the substituted phenylphosphates as mutual steroid-β-agonist prodrugs. The selected steroid analogs (described in Schemes IV and V) were alkylated with the benzylic mesylate of the protected phosphorylated β-agonist derivatives (3, 7 or 12 for salmeterol, albuterol or R-salmeterol, respectively) in the presence of a stoichiometric amount of sodium iodide in a polar, aprotic solvent like acetonitrile. It is beneficial to include the additional protection step prior to alkylation in the case of steroid substrates with an unprotected, primary 21-hydroxyl (see Scheme VII). The triphenylmethyl (Trt) moiety is a protective group of choice, compatible with the overall protection scheme and selectively introduced in mildly basic conditions (in presence of triethylamine and catalytic DMAP). In the final step, the intermediate quaternary ammonium (or in some cases sulfonium) salts were deprotected by mild acidolysis, advantageously by brief (up to 1 h) treatment with 4N HCl in dioxane yielding the target mutual prodrugs, e.g. 16 and 17, described in Examples 107 and 133, respectively.

### II. Enzymatic Activation of Substituted Phenylphosphate as Mutual Steroid-β-Agonist Prodrugs

Substituted phenylphosphates of the present invention (mutual prodrugs of steroids and β-agonists) are efficiently cleaved by alkaline phosphatase present in lungs, according to the process shown in Scheme VIII. This transformation occurs stepwise and consists of two distinct steps. First, the phosphate group is cleaved by alkaline phosphatase and the desphosphate intermediate forms. Then, the desphosphate intermediate slowly undergoes solvolysis by the addition of water to the benzylic position thereby simultaneously releasing the β-agonist and steroid.

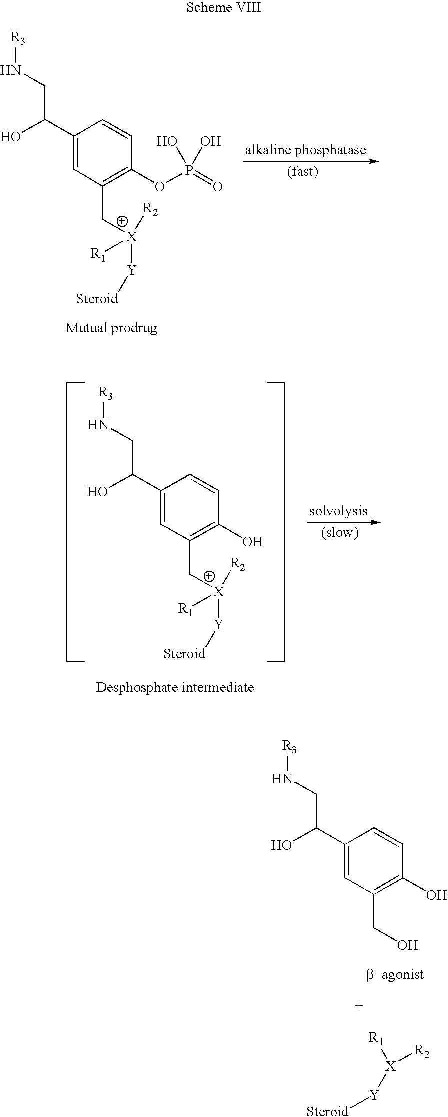

The detailed description of the enzymatic conversion of mutual prodrugs 16 and 17 is described in Examples 141-143 and depicted in FIGS. 1 and 2.

### III. Aerosol Delivery Devices

The use of the substituted phenylphosphates as mutual steroid-β-agonist prodrugs suitably formulated for liquid nebulization, or alternatively as a dry powder provides sufficient amount of the mutual prodrug to the lungs achieving a local therapeutic effect through releasing both bioactive components locally. Substituted phenylphosphate mutual prodrugs of the invention are suitable for aerosolization using jet, electronic, or ultrasonic nebulizers. They are also appropriate for delivery by dry powder or metered dose inhaler. Their solid form has long-term stability permitting the drug substance to be stored at room temperature.

The aerosol formulation comprises a concentrated solution of 1-10 mg/mL of pure substituted phenylphosphate as a mutual steroid-β-agonist prodrug or its pharmaceutically acceptable salt, dissolved in aqueous or aqueous-ethanolic solution having a pH between 4.0 and 7.5. Preferred pharmaceutically acceptable salts are inorganic acid salts including hydrochloride, hydrobromide, sulfate or phosphate salts as they may cause less pulmonary irritation. The therapeutic amount of the mutual prodrug is delivered to the lung endobronchial space by nebulization of a liquid aerosol or dry powder having an average mass median diameter between 1 to 5μ. A liquid formulation may require separation of a mutual prodrug salt from the appropriate diluent requiring reconstitution prior to administration because the long-term stability of the substituted phenylphosphate mutual prodrugs in aqueous solutions may not provide a commercially acceptable shelf life.

An indivisible part of this invention is a device able to generate aerosol from the formulation of the invention into aerosol particles predominantly in the 1-5μ size range. Predominantly, in this application, means that at least 70% but preferably more than 90% of all generated aerosol particles are within the 1-5μ size range. Typical devices include jet nebulizers, ultrasonic nebulizers, vibrating porous plate nebulizers, and energized dry powder inhalers.

A jet nebulizer utilizes air pressure to break a liquid solution into aerosol droplets. An ultrasonic nebulizer works by a piezoelectric crystal that shears a liquid into small aerosol droplets. A pressurized nebulization system forces solution under pressure through small pores to generate aerosol droplets. A vibrating porous plate device utilizes rapid vibration to shear a stream of liquid into appropriate droplet sizes. However, only some formulations of substituted phenylphosphate mutual prodrugs can be efficiently nebulized, as the devices are sensitive to the physical and chemical properties of the formulation. Typically, the formulations which can be nebulized, must contain small amounts of the substituted phenylphosphate mutual prodrugs, which are delivered in small volumes (50-250 μL) of aerosol.

### IV. Utility

The compounds of the invention are useful (in humans) for treating pulmonary inflammation and bronchoconstriction.

The amount of active ingredient that may be combined with the carrier materials to produce a single dosage form will vary depending upon the host treated and the particular mode of administration.

This small volume, high concentration formulation of substituted phenylphosphate steroid-β-agonist prodrug can be delivered as an aerosol and at efficacious concentrations to the respiratory tract in patients suffering from mild to severe asthma, bronchitis or chronic obstructive pulmonary disease (COPD). The solid dosage formulation is stable, readily manufactured and very cost effective. Furthermore, the formulation provides adequate shelf life for commercial distribution. The mutual prodrug masks the pharmacologic properties of steroids thus sore throat, fungal infections, dysphonia and other side effects in the oral pharyngeal cavity are completely eliminated. The prodrug also masks the β-agonist activity minimizing a chance for cardiovascular side-effects. Both drugs are released by enzymes present in lungs, specifically alkaline phosphatase, thereby releasing simultaneously the therapeutic amount of β-agonist and of a corticosteroid, at the site of inflammation and bronchoconstriction.

The foregoing may be better understood from the following examples, which are presented for the purposes of illustration and are not intended to limit the scope of the inventive concepts.

### Example 1

### Phosphorobromidic acid di-tert-butyl ester

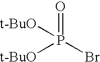

The title phosphorylating agent was prepared according to the modified conditions compared to those described by Gajda and Zwierzak (1976). By lowering the temperature of the reaction to 15° C. and decreasing the reaction time to 2.5 hours the title compound obtained in our hands had better purity then when applying the literature conditions (25° C. for 4 hours). The title phosphobromidate is unstable and was immediately used for the phosphorylation reactions (see Examples 4, 11 and 14).

Examples 2-6 illustrate the synthesis of the racemic phosphorylated derivative of salmeterol (see Scheme I).

### Example 2

### [2-Hydroxy-2-(4-hydroxy-3-hydroxymethyl-phenyl)-ethyl]-[6-(4-phenyl-butoxy)-hexyl-carbamic acid tert-butyl ester

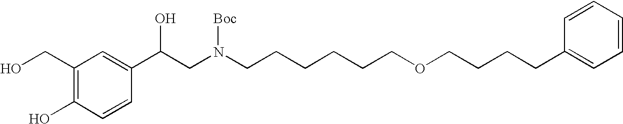

Commercially available salmeterol xinafoate (6.04 g, 10 mmol) and potassium carbonate (1.39 g, 10 mmol) were suspended with stirring in a 1,4-dioxane/water mixture (1:1, 80 mL). Then, di-t-butyl-dicarbonate (2.40 g, 11 mmol) dissolved in 1,4-dioxane (10 mL) was added dropwise while continuing stirring at room temperature. The TLC analysis after 30 minutes showed only traces of starting material. After 2 hours 1,4-dioxane was evaporated and the suspension formed was diluted with water and extracted twice with chloroform (125 mL total). Then, the organic layer was washed with saturated sodium bicarbonate, brine and dried over anhydrous magnesium sulfate. The crude material obtained after decantation and evaporation was purified by silica gel chromatography eluting with the ethyl acetate/hexane mixture (1:1). The title compound (4.61 g, 89%) was obtained as a glassy residue solidifying upon refrigeration.

LCMS: 100%, MNa⁺ 538.3 (exact mass 515.3 calcd for C₃₀H₄₅NO₆). Anal. Calc: C, 69.87; H, 8.80; N, 2.72. Found: C, 69.69; H, 8.64; N, 2.68.

### Example 3

### [2-(3-Formyl-4-hydroxy-phenyl)-2-hydroxy-ethyl]-[6-(4-phenyl-butoxy)-hexyl]-carbamic acid tert-butyl ester

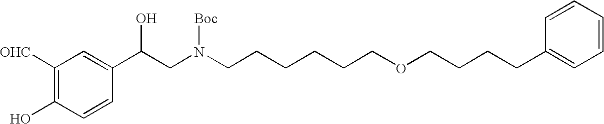

The N-Boc-salmeterol described in Example 2 (3.24 g, 6.28 mmol) was dissolved in chloroform (50 mL) and the activated manganese oxide (IV) (6.44 g, 85% w/w, 63 mmol) was added in portions with vigorous stirring. After 24 hours at room temperature the slurry was filtered through a pad of Celite, followed by the concentration of the filtrate combined with the chloroform washes. The crude residue thus obtained was purified by silica gel chromatography using ethyl acetate/hexane mixture (1:5) yielding the title aldehyde 1 (2.45 g, 77%). LCMS: 96%, MNa⁺ 536.3 (exact mass 513.3 calcd for C₃₀H₄₃NO₆).

### Example 4

### {2-[4-(Di-tert-butoxy-phosphoryloxy)-3-formyl-phenyl]-2-hydroxy-ethyl}-[6-(4-phenyl-butoxy)-hexyl]-carbamic acid tert-butyl ester

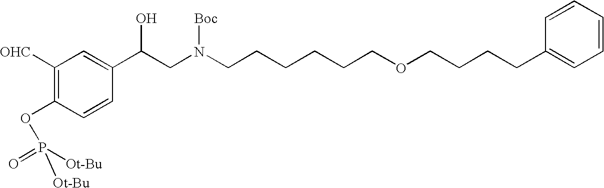

Aldehyde 1 (3.44 g, 6.69 mmol) was dissolved in anhydrous THF (10 mL), which was followed by adding DMAP (82 mg, 0.67 mmol) and DBU (1.11 mL, 7.4 mmol) with vigorous stirring under nitrogen. After cooling the reaction mixture to 0° C. the phosphobromidate described in Example 1 (2.19 g, 8 mmol) diluted with anhydrous THF (5 mL) was added dropwise over 15 minutes Stirring under nitrogen at 0° C. was continued for another 30 minutes, after which the TLC analysis showed the phosphorylation to be almost complete. After another 60 minutes the reaction mixture was concentrated, the residue was redissolved in ethyl acetate, washed 3 times with 10% citric acid, twice with 0.5N NaOH, brine and dried over anhydrous sodium sulfate. The organic phase was then filtered through a pad of basic alumina and the filtrate combined with ethyl acetate washes was concentrated in vacuo. The crude product was purified by silica gel chromatography using 30% ethyl acetate/1% triethylamine in hexane, yielding the title compound 2 (3.42 g, 72%) as a glassy residue.

³¹PNMR (CDCl₃): −15.107 ppm. LCMS: 100%, MNa⁺ 728.0 (exact mass 705.4 calcd for C₃₈H₆₀NO₉P). Anal. Calc: C, 64.66; H, 8.57; N, 1.98. Found: C, 64.09; H, 8.54; N, 2.02.

### Example 5

### {2-[4-(Di-tert-butoxy-phosphoryloxy)-3-hydroxymethyl-phenyl]-2-hydroxy-ethyl}-[6-(4-phenyl-butoxy)-hexyl]-carbamic acid tert-butyl ester

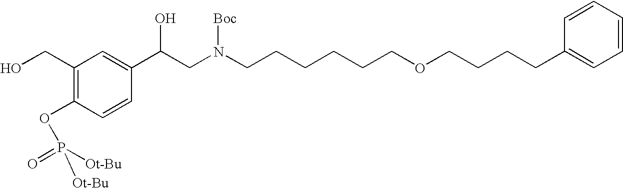

The phosphorylated aldehyde 2 (2.68, 3.8 mmol) was dissolved in anhydrous THF (10 mL) and the mixture was cooled to −78° C. Then, solid sodium borohydride (0.432 g, 11.4 mmol) was added in portions over 5 minutes with vigorous stirring under nitrogen, which was followed by adding methanol (1 mL). The reaction mixture was stirred allowing the temperature of the bath to increase to 0° C. over 4 hours (during which the TLC analysis showed consumption of the starting material). The reaction mixture was diluted with dichloromethane (50 mL), followed by careful quenching by adding 10% citric acid (20 mL) with vigorous stirring. The organic phase was separated, aqueous layer extracted with another portion of DCM and combined extracts were washed twice with saturated bicarbonate, brine, dried over anhydrous sodium sulfate, decanted and evaporated. The crude product was purified by chromatography using 40% ethyl acetate/1% triethylamine in hexane, yielding the title diol (2.01 g, 75%) as a colorless glassy residue.

¹H NMR (CDCl₃) selected signals: 7.17-7.41 (m, 8H), 4.92 (m, 1H), 4.62 (bs, 2H), 3.39 (q, 2H), 2.64 (t 2H), 1.62 (m, 4H), 1.54 (s, 9H), 1.52 (s, 9H), 1.49 (s, 9H), 1.115-1.49 (m, 8H). ³¹PNMR (CDCl₃): −13.060 ppm. LCMS: 99%, MNa⁺ 730.0 (exact mass 707.4 calcd for C₃₈H₆₂NO₉P). Anal. Calc: C, 64.48; H, 8.83; N, 1.98. Found: C, 64.70; H, 8.84; N, 1.90.

### Example 6

### Methanesulfonic acid 5-(2-{tert-butoxycarbonyl-[6-(4-phenyl-butoxy)-hexyl]-amino}-1-hydroxy-ethyl)-2-(di-tert-butoxy-phosphoryloxy)-benzyl ester

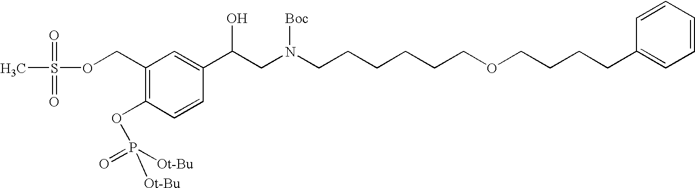

Compound 3 was synthesized by treating the diol described in Example 5 with the 1.1 equivalent of methanesulfonyl chloride in presence of 2 equiv. of 1,2,2,6,6-pentamethyl-piperidine (PMP) dissolved in anhydrous dichloromethane with vigorous stirring and cooling in water bath. The TLC monitoring showed the disappearance of the starting material after 30 minutes. After 1 hour the reaction mixture was concentrated in vacuo, followed with azeodrying by repeated evaporation with toluene. The crude mesylate 3 was immediately used for the quaternization (alkylation) of the steroid analogs (see Schemes VI and VII).

Examples 7-13 illustrate the synthesis of the racemic phosphorylated derivative of albuterol (see Scheme II).

### Example 7

### 2-tert-Butylamino-1-(2,2-dimethyl-4H-benzo[1,3]dioxin-6-yl)-ethanol

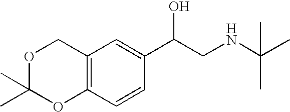

The title compound 4 was synthesized according to the procedure by Stevens (1999). Commercially available albuterol (salbutamol) suspended in dry acetone was treated with boron trifluoride etherate at 0° C. for 2 hours with vigorous stirring under nitrogen. The crude product was sufficiently pure (90%) to carry out the next step described in Example 8.

### Example 8

### tert-Butyl-[2-(2,2-dimethyl-4H-benzo[1,3]dioxin-6-yl)-2-hydroxy-ethyl]-carbamic acid tert-butyl ester

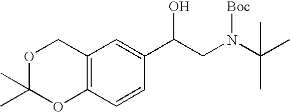

The O,O-isopropylidene protected albuterol (4) was dissolved in anhydrous THF (5 mL), which was followed by adding DMAP (0.1 equivalent) and triethylamine (1.1 equivalent) under nitrogen with stirring. Then, di-t-butyl dicarbonate (1.1 equivalent) dissolved in minimum amount of anhydrous THF was added via septum and the mixture stirred overnight at room temperature. Next day another equivalent of the acylating reagent was added and the mixture was further stirred with the TLC monitoring. After 48 hours THF was evaporated, the residue taken up in ethyl acetate and washed with 10% citric acid (3 times), saturated sodium bicarbonate (twice), brine and dried over magnesium sulfate. The crude product obtained after decantation and evaporation in vacuo was purified by siliga gel chromatography. The title compound 5 was obtained as a glassy residue in moderate yield.

LCMS: 95%, MH⁺ 380.3 (exact mass 379.3 calcd for C₂₁H₃₃NO₅).

### Example 9

### tert-Butyl-[2-hydroxy-2-(4-hydroxy-3-hydroxymethyl-phenyl)-ethyl]-carbamic acid tert-butyl ester

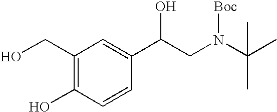

The title compound 6 can be prepared by refluxing of the protected derivative 5 in 80% (v/v) aqueous acetic acid. As soon as the TLC analysis shows the completeness of the isopropylidene hydrolysis the reaction mixture can be concentrated, redissolved in ethyl acetate washed with 10% citric acid, brine and dried over anhydrous magnesium sulfate. The crude product 6 should be of sufficient purity for the following oxidation.

### Example 10

### tert-Butyl-[2-(3-formyl-4-hydroxy-phenyl)-2-hydroxy-ethyl]-carbamic acid tert-butyl ester

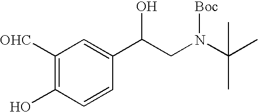

The title aldehyde can synthesized as described in Example 3, using the N-Boc-protected albuterol (6) as the starting material.

### Example 11

### tert-Butyl-{2-[4-(di-tert-butoxy-phosphoryloxy)-3-formyl-phenyl]-2-hydroxy-ethyl}-carbamic acid tert-butyl ester

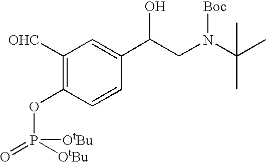

The title phosphorylated compound can be prepared analogously as described in Example 4, using the aldehyde described in Example 11 as the starting material.

### Example 12

### tert-Butyl-{2-[4-(di-tert-butoxy-phosphoryloxy)-3-hydroxymethyl-phenyl]-2-hydroxy-ethyl}-carbamic acid tert-butyl ester

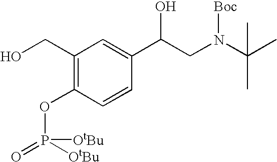

The title diol can be prepared by the borohydride reduction of the phosphorylated aldehyde described in Example 11, according to the procedure described in Example 5.

### Example 13

### Methanesulfonic acid 5-[2-(tert-butoxycarbonyl-tert-butyl-amino)-1-hydroxy-ethyl]-2-(di-tert-butoxy-phosphoryloxy)-benzyl ester

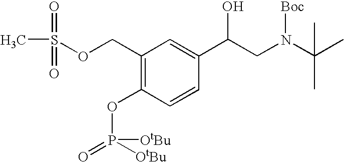

The title mesylate 7 can be prepared as described in Example 6, using the diol described in Example 12. The activated compound 7 can be used crude for the quaternization (alkylation) of the steroid moiety (see Scheme VI and VII).

Examples 14-21 illustrate the asymmetric synthesis of the phosphorylated/β-agonist derivative (see Scheme III).

### Example 14

### Phosphoric acid 4-bromo-2-formyl-phenyl ester di-tert-butyl ester

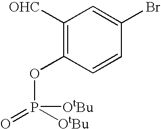

5-Bromosalicylaldehyde (8.04 g, 40 mmol) was phosphorylated analogously as described in Example 4, using DBU (6.58 mL, 44 mmol) and DMAP (0.489 g, 4 mmol) dissolved in anhydrous THF (50 mL) and cooled to 0° C. The phosphorylating agent was prepared as described in Example 1 (23.2 g, 85 mmol) and diluted with anhydrous THF (20 mL). The crude product was purified by chromatography (9% ethyl acetate+1% triethylamine in hexane) yielding analytically pure title aldehyde as a yellowish solid (11.51 g, 73%).

¹HNMR (CDCl₃): 10.35 (s, 1H), 7.99 (d, 1H, J=2.4 Hz), 7.67 (dd, 1H, J=8.8 Hz, 2.4 Hz), 7.41 (d, 1H, J=8.8 Hz), 1.51 (s, 18H). ³¹PNMR (CDCl₃): −15.239 ppm. LCMS: 99%, MNa⁺ 415 (exact mass 392.04 calcd for C₁₅H₂₂BrO₅P).

### Example 15

### Phosphoric acid 4-bromo-2-(tert-butyl-dimethyl-silanyloxymethyl)-phenyl ester di-tert-butyl ester

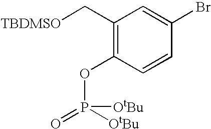

Aldehyde described in Example 14 was reduced to alcohol analogously as described in Example 5. The crude material solidified upon repeated evaporation with hexane and was sufficiently pure to continue the synthesis. The intermediate alcohol was converted to compound 8 by treatment with the slight excess of tert-butyldimethylsilyl chloride in DMF in presence of excess (5 equivalents) of imidazole. After the overnight reaction at room temperature the mixture was diluted with diethyl ether, washed extensively with 10% citric acid, brine and the organic phase was then dried with anhydrous magnesium sulfate, decanted and evaporated. The crude material was purified by chromatography using 10% ethyl acetate +1% triethylamine in hexane.

### Example 16

### 6-(4-Phenyl-butoxy)-hexylamine

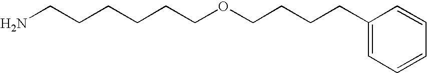

The title compound was prepared in a three-step process based on the procedure by Rong and Ruoho (1999). First, the alkoxide generated with NaH from 4-phenylbutanol was alkylated with 1,6-dibromohexane in presence of catalytic tetrabutylammonium bromide to give the bromoether (purified by vacuum distillation). Reaction of the bromoether with the excess (6 equivalents) of sodium azide in presence of 0.5 equivalent of sodium iodide in DMF at 80° C. produced the alkyl azide, purified by silica gel chromatography (ethyl acetate/hexane 1:30). The azide intermediate was reduced by hydrogenolysis in presence of 10% Pd/C catalyst, to give the title primary amine.

LCMS: 98%, MH+ 250.3 (exact mass 249.5 calcd for C₁₆H₂₇NO).

### Example 17

### Phosphoric acid di-tert-butyl ester 2-(tert-butyl-dimethyl-silanyloxymethyl)-4-vinyl-phenyl ester

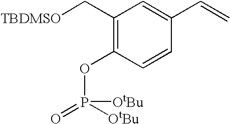

A two-neck, round bottomed flask, equipped with a reflux condenser was charged with the solution of compound 8 in a mixture of toluene (8 mL/mmol) and ethanol (1mL/mmol) followed by adding a degassed 20% solution of potassium carbonate (8 mL/mmol). The biphasic mixture was vigorously stirred for 1 hour while the stream of argon was passed through the flask. To this mixture, the trivinylboroxine-pyridine complex (1.5 equivalent) was added, followed by tricyclohexylphosphine (0.1 equivalent). The reaction mixture purged with argon once again for 30 minutes, then palladium (II) acetate (0.1 equivalent) was added, followed by vigorous stirring and heating under reflux under the positive pressure of argon for 4 hours. After that time the TLC analysis (chloroform/methanol 8:1) showed the complete consumption of starting material. The reaction mixture was diluted with ethyl acetate (3 times the original volume) and the organic phase was washed with water (3 times), 10% citric acid solution (twice) and brine and was dried over anhydrous MgSO₄. After filtration and evaporation of the solvent, the residue was purified by silica gel chromatography (ethyl acetate/hexanes 1:20 with 5% of triethylamine), yielding 80% of the desired olefin 9 as a viscous oil.

¹H NMR (CDCl₃): 7.52 (s, 1H), 7.27 (d, 1H), 7.19 (d, 1H), 6.67 (dd, 1H), 5.66 (d, 1H), 5.17 (d, 1H), 4.71 (s, 2H), 1.48 (s, 18H), 0.95 (s, 9H), 0.10 (s, 6H). ³¹P NMR (CDCl₃): −14.18 ppm. LCMS: 95%, MNa+ 479 (exact mass 456.3 calcd for C₂₃H₄₁O₅PSi).

### Example 18

### Phosphoric acid di-tert-butyl ester 2-(tert-butyl-dimethyl-silanyloxymethyl)-(S)-4-oxiranyl-phenyl ester

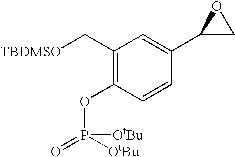

Compound 9 was dissolved in a biphasic mixture of methylene chloride (5 mL/mmol) and phosphate buffer (3 mL/mmol), which was followed by addition of sodium hypochlorite (0.2 mL/mmol), N-methylmorpholine-N-oxide (0.25 equivalent) and the S,S-version of Jacobsen's (Jacobsen, 1991) catalyst [(S,S)-(+) N,N′-Bis(3,5-di-tert-butylsalicylidene)-1,2-cyclohexanediaminomanganese(III) chloride; 0.1 equivalent]. The reaction mixture was stirred for 4 hours at 30° C., after which time the TLC analysis (chloroform/methanol 8:1) revealed the complete consumption of the starting material. The reaction mixture was transferred into the separating funnel and allowed to settle. The aqueous layer was discarded and the organic phase was washed with water (twice), 10% citric acid solution (twice), brine and dried over anhydrous MgSO₄. After filtration and evaporation the residue was purified by silica gel chromatography (ethyl acetate/hexanes 1:10 with 5% of triethylamine). The title compound 10 was obtained with 62% yield and the enantiomeric excess exceeding 90% (as determined by APCI-LCMS on a column Daicel Chiralpak IA from Chiral Technologies).

¹H NMR (CDCl₃): 7.41 (s, 1H), 7.26 (d, 1H), 7.06 (d, 1H), 4.77 (s, 2H), 3.70 (s, 1H), 3.08 (dd, 1H), 2.74 (dd, 1H), 1.46 (s, 18H), 0.92 (s, 9H), 0.08 (s, 6H). ³¹P NMR (CDCl₃): −14.16 ppm. LCMS: 97%, MNa+ 495.3 (exact mass 472.3 calcd for C₂₃H₄₁O₆PSi).

### Example 19

### Phosphoric acid di-tert-butyl ester 2-(tert-butyl-dimethyl-silanyloxymethyl)-4-{(R)-1-hydroxy-2[6-(4-phenyl-butoxy)-hexylamino]-ethyl}-phenyl ester

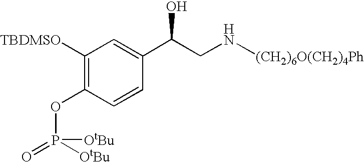

The title derivative 11 can be prepared by the nucleophilic opening of the chiral epoxide 10 by reacting with the slight excess of 6-(4-phenylbutoxy)-hexylamine (described in Example 16) in 95% aqueous ethanol applying gentle heating (40° C. should not be exceeded to avoid the thermal monodeprotection of the phosphate diester). As soon as the TLC analysis shows the consumption of the starting epoxide the reaction mixture can be evaporated in vacuo and the crude product used directly in the next step (Example 20).

### Example 20

### {2-[3-(tert-Butyl-dimethyl-silanyloxymethyl)-4-(di-tert-butoxy-phosphoryloxy)-phenyl]-(R)-2-hydroxy-ethyl}-[6-(4-phenyl-butoxy)-hexyl]-carbamic acid tert-butyl ester

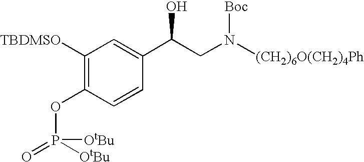

The title compound can be prepared by the Boc protection of the secondary amine 11 (described in Example 19) applying the analogous procedure as described in Example 8, except that lower excess of the di-t-butyl dicarbonate and shorter reaction time (4-16 h) can be used due to higher reactivity of the unhindered secondary amine.

### Example 21

### Methanesulfonic acid 5-(2-{tert-butoxycarbonyl-[6-(4-phenyl-butoxy)-hexyl]-amino}-(R)-1-hydroxy-ethyl)-2-(di-tert-butoxy-phosphoryloxy)-benzyl ester

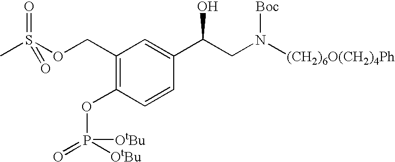

The protected derivative described in Example 20 can be treated with 1M solution of TBAF in THF at room temperature. As soon as the TLC analysis shows the complete deprotection (usually 1-2 hours) the crude product obtained after evaporation of the solvent can be purified by chromatography using 40% ethyl acetate +1% triethylamine in hexane.

The title compound 12 can be synthesized by treating thus obtained diol with 1.1 equivalent of methanesulfonyl chloride in presence of 2 equivalents of 1,2,2,6,6-pentamethylpiperidine dissolved in dichloromethane at room temperature, analogously as described in Example 6. The crude mesylate 12 can be immediately used for the quaternization (alkylation) of the steroid analogs (see Scheme VI and VII).

Examples 22-55 describe the synthesis of steroid analogs according to Scheme IV.

### Example 22

### 16,17-[(Cyclohexylmethylene)bis(oxy)]-11,21-dihydroxypregna-1,4-diene-3,20-dione[11β,16α(R)]

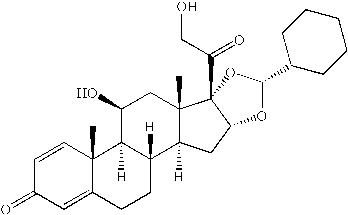

Desonide (4.16 g; 10 mmol) was dissolved in 1-nitropropane (14 mL) and cooled to 0° C. To this solution, 70% perchloric acid (2.6 mL, 30 mmol) was added dropwise over 5 minutes, followed by cyclohexylcarboxaldehyde (1.44 mL, 12 mmol) and the reaction mixture was stirred for the following 3 hours at 0° C. and then the reaction mixture was allowed to warm up overnight to room temperature. The TLC analysis (ethyl acetate/hexanes 1:1) indicated complete consumption of the starting material. The reaction mixture was diluted with ethyl acetate (10 times the volume) and washed with saturated sodium bicarbonate solution (3 times), twice with water and brine. The organic solution was then dried with anhydrous magnesium sulfate, filtered and the solvent was removed in vacuo. The crude product was purified by silica gel chromatography (ethyl acetate/hexane 1:2) and finally recrystallized from ethyl acetate/hexane yielding the title compound as a white solid (59%).

LCMS: 97%, MH+ 471.3 (exact mass 470.3 calcd for C₂₈H₃₈O₆). Optical rotation [α_{D}]=+76.0 deg (c 0.5; MeOH).

The 2D NMR study confirmed the connectivities and the R-configuration at the C-22 atom (epimeric purity was >95% within precision of the NMR method).

### Example 23

### 16,17-[(Cyclohexylmethylene)bis(oxy)]-9-fluoro-11,21-dihydroxypregna-1,4-diene-3,20-dione[11β,16α(R)]

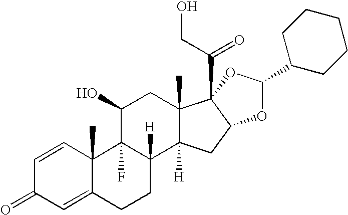

The title compound was prepared as described in Example 22, substituting desonide with triamcinolone acetonide. The desired acetal was obtained as a white solid in 48% yield.

¹⁹FNMR (CDCl₃): −165.3 ppm (dd, J=9.6 Hz, J=31.6 Hz). LCMS: 98%, MH⁺ 489.3 (exact mass 488.3 calcd for C₂₈H₃₇FO₆). Anal. Calc: C, 68.83; H, 7.63. Found: C, 68.81; H, 7.61. Optical rotation [α_{D}]=+84.0 deg (c 0.5; MeOH).

According to the ¹⁹FNMR analysis the undesired 22S-epimer was not formed.

### Example 24

### 16,17-[(Cyclohexylmethylene)bis(oxy)]-11-hydroxy-21-methanesulfonyloxy-pregna-1,4-diene-3,20-dione[11β,16α(R)]

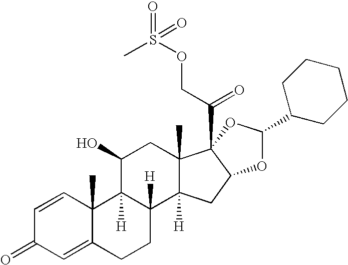

To a solution of steroid described in Example 22 (5 mL of DCM/mmol) was added 1,2,2,6,6-pentamethylpiperidine (2 equivalents) followed by the dropwise addition of methanesulfonyl chloride (1.1 equivalent) with vigorous stirring and cooling in the water bath. The TLC analysis revealed no starting material usually after 3-4 hours. After diluting with dichloromethane the reaction mixture was transferred to the separating funnel and washed with 10% citric acid (3 times), twice with saturated sodium bicarbonate solution, then with brine and finally dried over anhydrous magnesium sulfate. The drying agent was filtered and the solvent was removed in vacuo to yield the crude product which was triturated with diethyl ether inducing crystallization. The precipitate thus formed was filtered off, washed thoroughly with ether and dried, yielding the mesylate with purity sufficient for further synthesis.

¹H NMR (CDCl₃): 7.230 (d, 1H), 6.291 (d, 1H), 6.029 (s, 1H), 4.992 (AB, 2H), 4.849 (bs, 1H), 4.509 (bs, 1H), 4.302 (d, 1H), 3.242 (s, 3H), 2.557 (dt, 1H), 2.330 (m, 1H), 2.170 (m, 1H), 2.070 (m, 1H), 1.722 (m, 13H), 1.447 (s, 3H), 1.339 (m, 6H), 0.855 (s, 3H). LCMS: 97%, MH⁺ 549.3 (exact mass 548.3 calcd for C₂₉H₄₀O₈S). Optical rotation [α]_{D}=+75.1 (c 0.5; MeOH).

### Example 25

### 16,17-[(Cyclohexylmethylene)bis(oxy)]-9-fluoro-11-hydroxy-21-methanesulfonyloxy-pregna-1,4-diene-3,20-dione[11β,16α(R)]

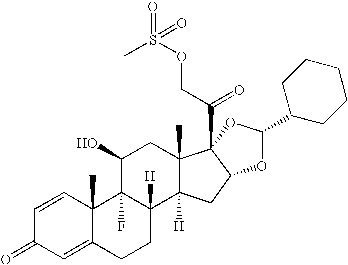

The title mesylate was synthesized as described in Example 24 using the steroid acetal described in Example 23.

¹H NMR (CDCl₃): 7.211 (d, 1H), 6.359 (dd, 1H), 6.139 (s, 1H), 5.009 (AB, 2H), 4.855 (d, 1H), 4.431 (m, 1H), 4.350 (d, 1H), 3.245 (s, 3H), 2.621 (dt, 1H), 2.402 (m, 4H), 2.155 (dt, 1H), 1.845 (m, 1H), 1.645 (m, 9H), 1.54 (s, 3H), 1.115 (m, 6H), 0.96 (s, 3H). ¹⁹F NMR (CDCl₃): −166.04 ppm (dd, J=9.6 Hz, J=31.6 Hz). LCMS: 98%, MH⁺ 567.3 (exact mass 566.3 calcd for C₂₉H₃₉FO₈S). Optical rotation [α]_{D}=+99.4 (c 0.5; MeOH).

### Example 26

### 16,17-[(Cyclohexylmethylene)bis(oxy)]-11-hydroxy-21-(4-methylpiperazyn-1-yl)-pregna-1,4-diene-3,20-dione[11β,16α(R)]

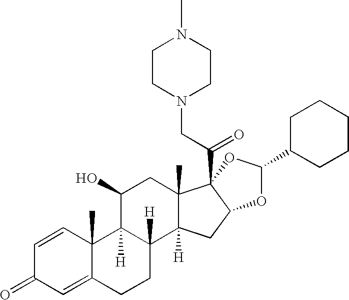

To a mixture of the mesylate described in Example 24 (1 equivalent), 4-methylpiperazine (3 equivalents) and finely powdered anhydrous potassium carbonate (2 equivalents) the anhydrous acetonitrile (5 mL/mmol) was added and the resulting suspension was stirred while heating at 60° C. overnight. Then the reaction mixture was diluted with ethyl acetate (10 times the volume) and washed twice with water, 10% citric acid, saturated sodium bicarbonate and finally with brine. After drying over anhydrous magnesium sulfate, filtration and evaporation the crude material was purified by silica gel chromatography using a mixture of ethyl acetate/methanol (10:1), yielding the title compound (42%) as a white solid.

¹H NMR (CDCl₃): 7.246 (d, 1H), 6.289 (dd, 1H), 6.029 (s, 1H), 4.888 (d, 1H), 4.500 (m, 1H), 4.255 (d, 1H), 3.402 (AB, 2H), 2.561 (m, 8H), 2.328 (s, 3H), 1.737 (m, 5H), 1.671 (m, 3H), 1.561 (m, 3H), 1.446 (s, 3H), 1.155 (m, 11H), 0.902 (s, 3H), 0.819 (m, 1H).

LCMS: 99%, MH⁺ 553.4 (exact mass 552.4 calcd for C₃₃H₄₈N₂O₅). Optical rotation [α_{D}]=+89.6° (c 0.5; MeOH).

### Example 27

### 16,17-[(Cyclohexylmethylene)bis(oxy)]-9-fluoro-11-hydroxy-21-(4-methylpiperazyn-1-yl)-pregna-1,4-diene-3,20-dione[11β,16α(R)]

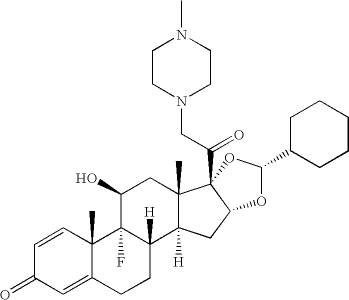

The mesylate described in Example 25 was reacted with 4-methylpiperazine as described in Example 26. The crude product was purified by chromatography (ethyl acetate/methanol 10:1), followed by recrystallization from chloroform/hexane, yielding the title compound 13.

¹H NMR (CDCl₃): 7.211 (d, 1H), 6.365 (d, 1H), 6.135 (s, 1H), 4.895 (d, 1H), 4.295 (d, 1H), 3.412 (AB, 2H), 2.620 (dt, 1H), 2.542 (m, 6H), 2.410 (m, 4H), 2.304 (s, 3H), 2.140 (dt, 1H), 1.840 (m, 1H), 1.697 (m, 12H), 1.548 (s, 3H), 1.120 (m, 6H), 0.907 (s, 3H). ¹⁹FNMR (CDCl₃): −165.4 ppm (dd, J=9.6 Hz, J=31.6 Hz). LCMS: 99%, MH⁺ 571.3 (exact mass 570.4 calcd for C₃₃H₄₇FN₂O₅). Optical rotation [α_{D}]=+89.6° (c 0.5; MeOH).

### Example 28

### 16,17-[(Cyclohexylmethylene)bis(oxy)]-11-hydroxy-21-(4-morpholin-1-yl)-pregna-1,4-diene-3,20-dione[11β,16α(R)]

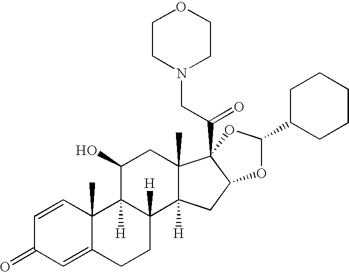

The title compound was prepared analogously as described in Example 26, substituting 4-methylpiperazine with morpholine.

¹H NMR (CDCl₃): 7.246 (d, 1H), 6.291 (dd, 1H), 6.036 (s, 1H), 4.882 (d, 1H), 4.511 (bs, 1H), 4.268 (d, 1H), 3.780 (t, 4H), 3.399 (AB, 2H), 2.575 (m, 3H), 2.474 (m, 1H), 2.355 (m, 1H), 2.080 (m, 3H), 1.736 (m, 12H), 1.448 (s, 3H), 1.275 (m, 3H), 1.221 (m, 4H), 0.907 (s, 3H). LCMS: 100%, MH⁺ 540.4 (exact mass 539.4 calcd for C₃₂H₄₅NO₆). Optical rotation [α_{D}]=+61.0° (c 0.5; MeOH).

### Example 29

### 16,17-[(Cyclohexylmethylene)bis(oxy)]-11-hydroxy-21-(1-piperidin-1-yl)-pregna-1,4-diene-3,20-dione[11β,16α(R)]

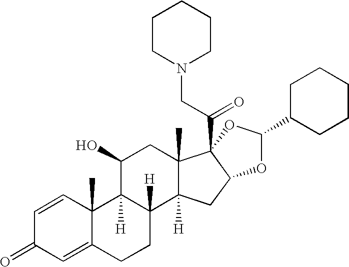

The title compound was prepared analogously as described in Example 26, substituting 4-methylpiperazine with piperidine. The final purification of the product was accomplished by chromatography on silica-gel using ethyl acetate as an eluent followed by the crystallization from dichloromethane/diethyl ether.

¹H NMR (CDCl₃): 7.246 (d, 1H), 6.290 (dd, 1H), 6.032 (s, 1H), 4.898 (d, 1H), 4.502 (s, 1H), 4.252 (d, 1H), 3.360 (AB, 2H), 2.553 (dt, 1H), 2.480 (bs, 1H), 2.358 (m, 3H), 2.078 (m, 3H), 1.684 (m, 12H), 1.550 (m, 3H), 1.446 (s, 3H), 1.159 (m, 10H), 0.907 (s, 3H). LCMS: 98%, MH⁺ 538.4 (exact mass 537.4 calcd for C₃₃H₄₇NO₅). Optical rotation [α_{D}]=+98.9 (c 0.5; MeOH).

### Example 30

### 16,17-[(Cyclohexylmethylene)bis(oxy)]-11-hydroxy-21-(pyrrolidin-1-yl)-pregna-1,4-diene-3,20-dione[11β,16α(R)]

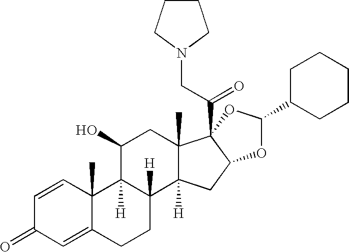

The title compound can be prepared analogously as described in Example 26, substituting 4-methylpiperazine with pyrrolidine.

### Example 31

### 16,17-[(Cyclohexylmethylene)bis(oxy)]-11-hydroxy-21-(N,N-diethylamino)-pregna-1,4-diene-3,20-dione[11β,16α(R)]

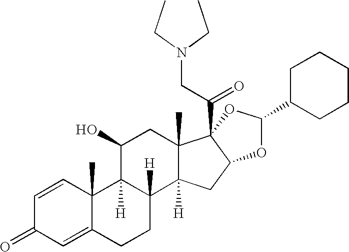

The title compound can be prepared analogously as described in Example 26, substituting 4-methylpiperazine with diethylamine.

### Example 32

### 16,17-[(Cyclohexylmethylene)bis(oxy)]-11-hydroxy-21-(N,N-dimethylamino)-pregna-1,4-diene-3,20-dione[11β,16α(R)]

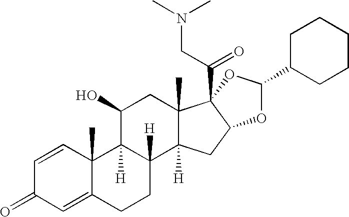

The title compound was prepared analogously as described in Example 26, substituting 4-methylpiperazine with dimethylamine (2M solution in THF).

¹H NMR (CDCl₃): 7.261 (d, 1H), 6.306 (dd, 1H), 6.053 (s, 1H), 4.922 (d, 1H), 4.522 (m, 1H), 4.275 (d, 1H), 3.371 (AB, 2H), 2.573 (dt, 1H), 2.333 (s, 6H), 2.114 (m, 4H), 1.683 (m, 10H), 1.467 (s, 3H), 1.180 (m, 8H), 0.930 (s, 3H). LCMS: 95%, MH⁺ 498.4 (exact mass 497.4 calcd for C₃₀H₄₃NO₅). Optical rotation [α_{D}]=+74.8° (c 0.5; MeOH).

### Example 33

### 16,17-[(Cyclohexylmethylene)bis(oxy)]-11-hydroxy-21-(4-methylhomopiperazin-1-yl)-pregna-1,4-diene-3,20-dione[11β,16α(R)]

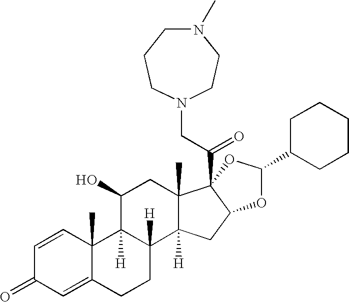

The title compound can be prepared analogously as described in Example 26, substituting 4-methylpiperazine with 4-methylhomopiperazine.

### Example 34

### 16,17-[(Cyclohexylmethylene)bis(oxy)]-9-fluoro-11-hydroxy-21-(4-morpholin-1-yl)-pregna-1,4-diene-3,20-dione[11β,16α(R)]

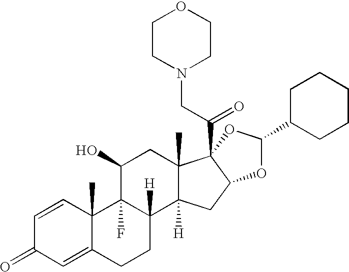

The title compound was prepared analogously as described in Example 27, substituting 4-methylpiperazine with morpholine.

¹H NMR (CDCl₃): 7.182 (d, 1H), 6.351 (d, 1H), 6.134 (s, 1H), 4.891 (d, 1H), 4.430 (m, 1H), 4.310 (d, 1H), 3.782 (t, 4H), 3.422 (AB, 2H), 2.609 (m, 3H), 2.451 (m, 5H), 1.850 (m, 2H), 1.650 (m, 10H), 1.541 (s, 3H), 1.142 (m, 6H), 0.914 (s, 3H). ¹⁹F NMR (CDCl₃): −165.86 ppm. LCMS: 96%, MH⁺ 558.4 (exact mass 557.4 calcd for C₃₂H₄₄FNO₆). Optical rotation [α_{D}]=+78.9° (c 0.5; MeOH).

### Example 35

### 16,17-[(Cyclohexylmethylene)bis(oxy)]-9-fluoro-11-hydroxy-21-(1-piperidin-1-yl)-pregna-1,4-diene-3,20-dione[11β,16α(R)]

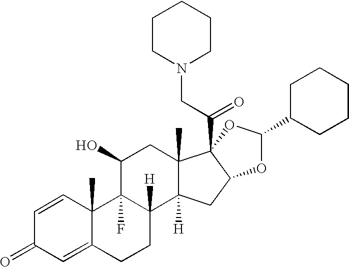

The title compound was prepared analogously as described in Example 27, substituting 4-methylpiperazine with piperidine. The crude product was purified by chromatography on silica-gel using methanol in ethyl acetate (0 to 10% gradient elution), followed by crystallization from ethyl acetate/diethyl ether.

¹H NMR (CDCl₃): 7.204 (d, 1H), 6.371 (dd, 1H), 6.151 (s, 1H), 4.911 (d, 1H), 4.449 (m, 1H), 4.300 (d, 1H), 3.389 (AB, 2H), 2.495 (m, 8H), 1.751 (m, 17H), 1.561 (s, 3H), 1.157 (m, 6H), 0.932 (s, 3H), 0.845 (m, 1H). ¹⁹F NMR (CDCl₃): −165.81 ppm. LCMS: 98%, MH⁺ 556.4 (exact mass 555.4 calcd for C₃₃H₄₆FNO₅). Optical rotation [α_{D}]=+75.1° (c 0.5; CHCl₃).

### Example 36

### 16,17-[(Cyclohexylmethylene)bis(oxy)]-9-fluoro-11-hydroxy-21-(1-pyrrolidin-1-yl)-pregna-1,4-diene-3,20-dione[11β,16α(R)]

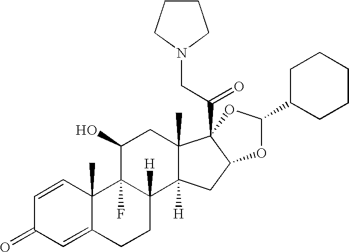

The title compound can be prepared analogously as described in Example 27, substituting 4-methylpiperazine with pyrrolidine.

### Example 37

### 16,17-[(Cyclohexylmethylene)bis(oxy)]-9-fluoro-11-hydroxy-21-(N,N-diethylamino)-pregna-1,4-diene-3,20-dione[11β,16α(R)]

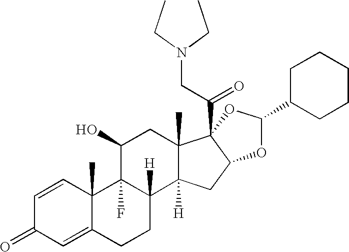

The title compound can be prepared analogously as described in Example 27, substituting 4-methylpiperazine with diethylamine.

### Example 38

### 16,17-[(Cyclohexylmethylene)bis(oxy)]-9-fluoro-11-hydroxy-21-(N,N-dimethylamino)-pregna-1,4-diene-3,20-dione[11β,16α(R)]

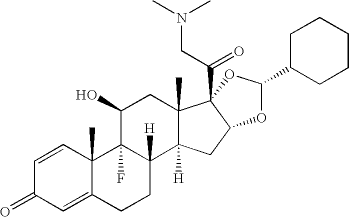

The title compound was prepared analogously as described in Example 27, substituting 4-methylpiperazine with dimethylamine (2M solution in THF).

¹H NMR (CDCl₃): 7.195 (d, 1H), 6.349 (dd, 1H), 6.132 (s, 1H), 4.905 (d, 1H), 4.414 (d, 1H), 4.298 (d, 1H), 3.368 (AB, 2H), 2.626 (dt, 1H), 2.410 (m, 3H), 2.331 (s, 6H), 2.151 (dt, 1H), 1.851 (m, 1H), 1.715 (m, 5H), 1.600 (m, 6H), 1.542 (s, 3H), 1.152 (m, 5H), 0.941 (s, 3H). ¹⁹F NMR (CDCl₃): −165.81 ppm. LCMS: 98%, MH⁺ 516.4 (exact mass 515.4 calcd for C₃₀H₄₂FNO₅). Optical rotation [α_{D}]=+74.6° (c 0.5; MeOH).

### Example 39

### 16,17-[(Cyclohexylmethylene)bis(oxy)]-9-fluoro-11-hydroxy-21-(4-methylhomopiperazin-1-yl)-pregna-1,4-diene-3,20-dione[11β,16α(R)]

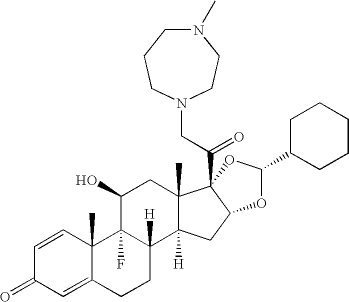

The title compound can be prepared analogously as described in Example 27, substituting 4-methylpiperazine with 4-methylhomopiperazine.

### Example 40

### 16,17-[(Cyclohexylmethylene)bis(oxy)]-11-hydroxy-21-(4-fluoropiperidin-1-yl)-pregna-1,4-diene-3,20-dione[11β,16α(R)]

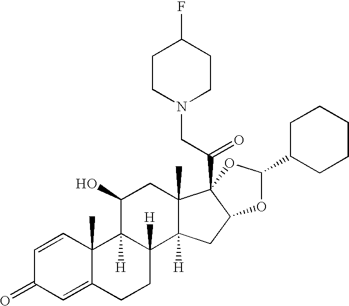

The title compound was prepared analogously as described in Example 26, substituting 4-methylpiperazine with 4-fluoropiperidine hydrochloride. Final purification was accomplished by the preparative HPLC, yielding the title compound as monotrifluoroacetate.

¹⁹FNMR (CDCl₃): −75.573 (s, 3F), −188.882 (m, 1F). LCMS: 99%, MH⁺ 556.4 (exact mass 555.3 calcd for C₃₃H₄₆FNO₅).

### Example 41

### 16,17-[(Cyclohexylmethylene)bis(oxy)]-9-fluoro-11-hydroxy-21-(4-fluoropiperidin-1-yl)-pregna-1,4-diene-3,20-dione[11β,16α(R)]

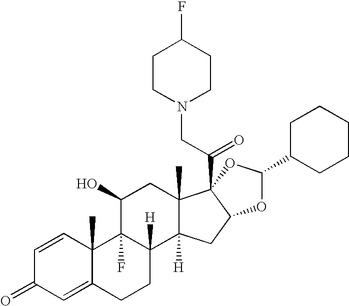

The title compound was prepared analogously as described in Example 27, substituting 4-methylpiperazine with 4-fluoropiperidine hydrochloride. Final purification was accomplished by the preparative HPLC, yielding the title compound as monotrifluoroacetate.

¹⁹F NMR (CDCl₃): −75.592 (s, 3F), −166.933 (dd, 1F), −188.915 (m, 1F). LCMS: 100%, MH⁺ 574.4 (exact mass 573.3 calcd for C₃₃H₄₅F₂NO₅).

### Example 42

### 16,17-[(Cyclohexylmethylene)bis(oxy)]-11-hydroxy-21-(azetidin-1-yl)-pregna-1,4-diene-3,20-dione[11β,16α(R)]

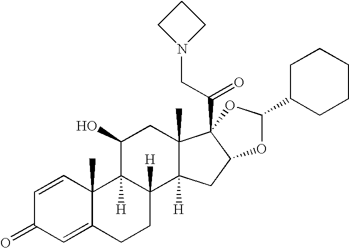

The title compound can be prepared analogously as described in Example 26, substituting 4-methylpiperazine with azetidine.

### Example 43

### 16,17-[(Cyclohexylmethylene)bis(oxy)]-9-fluoro-11-hydroxy-21-(azetidin-1-yl)-pregna-1,4-diene-3,20-dione[11β,16α(R)]

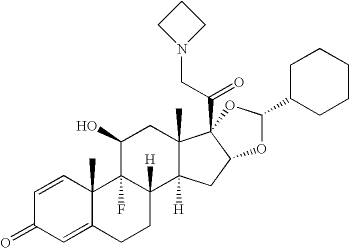

The title compound was prepared analogously as described in Example 27, substituting 4-methylpiperazine with azetidine. Final purification was accomplished by the preparative HPLC, yielding the product as a monotrifluoroacetate.

¹H NMR (DMSO-d₆): 10.135 (b, 1H), 7.357 (d, 1H), 6.251 (dd, 1H), 6.025 (bs, 1H), 5.600 (d, 1H), 4.605-4.690 (m, 2H), 4.470 (d, 1H), 4.370-4.420 (m, 1H), 3.950-4.220 (m, 6H), 2.537-2.670 (m, 1H), 2.220-2.490 (m, 3H), 1.907-2.040 (m, 2H), 1.554-1.820 (m, 10H), 1.481 (s, 3H), 1.038-1.410 (m, 6H), 0.826 (s, 3H). ¹⁹F NMR (DMSO-d₆): −73.526 (s, 3F); −165.106 (dd, 1F). LCMS: 98%, MH⁺ 528.4 (exact mass 527.4 calcd for C₃₁H₄₂FNO₅).

### Example 44

### 16,17-[(Cyclohexylmethylene)bis(oxy)]-11-hydroxy-21-(imidazol-1-yl)-pregna-1,4-diene-3,20-dione[11β,16α(R)]

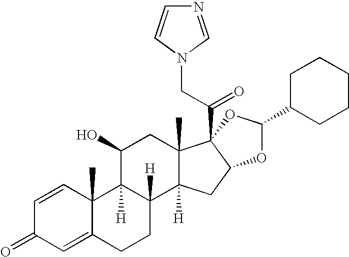

The title compound was prepared as described in Example 26, substituting 4-methylpiperazine with imidazole. The crude product was purified by silica gel chromatography using ethyl acetate as an eluent, followed by the crystallization from dichloromethane/diethyl ether.

¹H NMR (CDCl₃): 7.692 (s, 1H), 7.384 (s, 1H), 7.277 (d, 2H), 7.106 (d, 2H), 6.849 (s, 1H), 6.298 (d, 1H), 6.041 (s, 1H), 4.874 (d, 1H), 4.815 (AB, 2H), 4.551 (bs, 1H), 4.32 (d, 1H), 2.574 (dt, 1H), 2.354 (dd, 1H), 2.185 (m, 1H), 2.115 (m, 2H), 1.175 (m, 5H), 1.651 (m, 5H), 1.475 (s, 3H), 1.250 (m, 2H), 1.116 (m, 3H), 0.946 (s, 3H). LCMS: 100%, MH⁺ 521.4 (exact mass 520.4 calcd for C₃₁H₄₀N₂O₅). Optical rotation [α]_{D}=+112.3 (c 0.5; MeOH).

### Example 45

### 16,17-[(Cyclohexylmethylene)bis(oxy)]-9-fluoro-11-hydroxy-21-(imidazol-1-yl)-pregna-1,4-diene-3,20-dione[11β,16α(R)]

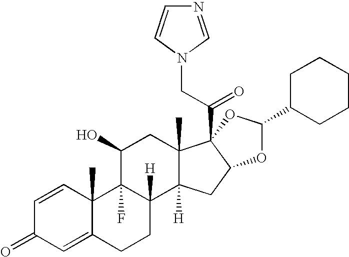

The title compound was prepared as in Example 27, substituting 4-methylpiperazine with imidazole. The crude product was purified by silica gel chromatography using methanol in ethyl acetate (0 to 10% gradient elution), followed by crystallization from dichloromethane/diethyl ether.

¹H NMR (CDCl₃): 7.373 (s, 1H), 7.280 (d, 1H), 7.082 (s, 1H), 6.875 (s, 1H), 6.345 (d, 1H), 6.141 (s, 1H), 4.880 (d, 1H), 4.831 (AB, 2H), 4.461 (m, 1H), 4.375 (d, 1H), 2.641 (dt, 1H), 2.495 (dt, 1H), 2.410 (m, 2H), 1.870 (m, 2H), 1.740 (m, 4H), 1.620 (m, 6H), 1.593 (s, 3H), 1.205 (m, 3H), 1.110 (m, 3H), 0.960 (s, 3H). ¹⁹F NMR (CDCl₃): −166.03 ppm.

LCMS: 97%, MH⁺ 539.4 (exact mass 538.4 calcd for C₃₁H₃₉FN₂O₅). Optical rotation [α]_{D}=+101.6 (c 0.5; CHCl₃).

### Example 46

### 16,17-[(Cyclohexylmethylene)bis(oxy)]-11-hydroxy-21-(pyridin-4-yl-thio)-pregna-1,4-diene-3,20-dione[11β,16α(R)]

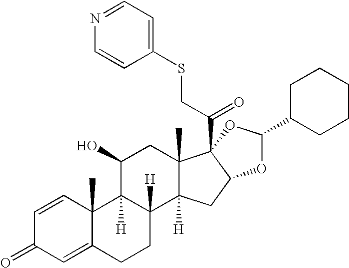

The title compound can be prepared as described in Example 26, substituting 4-methylpiperazine with pyridine-4-thiol.

### Example 47

### 16,17-[(Cyclohexylmethylene)bis(oxy)]-9-fluoro-11-hydroxy-21-(pyridin-4-yl-thio)-pregna-1,4-diene-3,20-dione[11β,16α(R)]

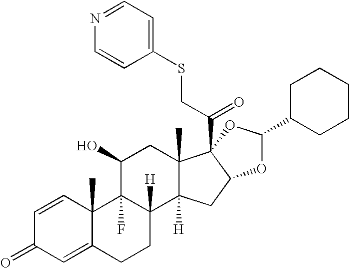

The title compound was prepared as in Example 27, substituting 4-methylpiperazine with pyridine-4-thiol. The crude product was purified by silica gel chromatography using gradient elution starting from 33% ethyl acetate in hexanes to 100% ethyl acetate.

¹H NMR (DMSO-d₆): 8.388 (dd, 2H), 7.270-7.310 (m, 3H), 6.238 (dd, 1H), 6.022 (bs, 1H), 5.434 (dd, 1H), 4.754 (bt, 1H), 4.465 (s, 1H), 4.314 (AB, 2H), 4.197-4.224 (m, 1H), 2.617 (dt, 1H), 2.315-2.413 (b, 2H), 2.132-2.166 (m, 1H), 1.984-2.062 (m, 1H), 1.784-1.826 (m, 2H), 1.658-1.720 (m, 4H), 1.540-1.612 (m, 4H), 1.484 (s, 3H), 1.060-1.393 (m, 6H), 0.828 (s, 3H). ¹⁹F NMR (DMSO-d₆): −165.392. LCMS: 98%, MH⁺ 582.4 (exact mass 581.4 calcd for C₃₃H₄₀FNO₅S).

### Example 48

### 16,17-[(Cyclohexylmethylene)bis(oxy)]-11-hydroxy-21-(pyridin-2-yl-thio)-pregna-1,4-diene-3,20-dione[11β,16α(R)]

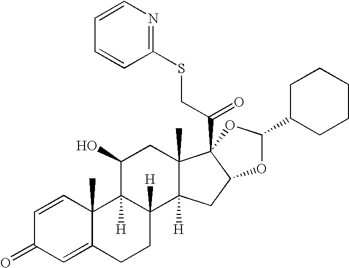

The title compound can be prepared as described in Example 26, substituting 4-methylpiperazine with pyridine-2-thiol.

### Example 49

### 16,17-[(Cyclohexylmethylene)bis(oxy)]-9-fluoro-11-hydroxy-21-(pyridin-2-yl-thio)-pregna-1,4-diene-3,20-dione[11β,16α(R)]

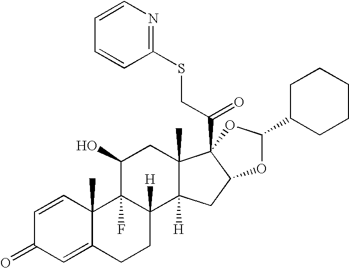

The title compound can be prepared as described in Example 27, substituting 4-methylpiperazine with pyridine-2-thiol, except for the modification in the purification procedure. The thick precipitate formed in reaction mixture was filtered off and washed several times with water and then with diethyl ether to yield the first crop of the desired product. The ethereal washings were collected, dried with anhydrous magnesium sulfate and concentrated to the small volume. The copious amount of hexanes was then added and the second crop of the precipitated product was collected by filtration.

¹H NMR (DMSO-d₆): 8.373 (d, 1H), 7.639 (dt, 1H), 7.308-7.369 (m, 2H), 7.116 (dd, 1H), 6.243 (dd, 1H), 6.025 (bs, 1H), 5.50 (d, 1H), 4.715 (d, 1H), 4.553 (d, 1H), 4.302 (AB, 2H), 4.201-4.299 (m, 1H), 2.620 (dt, 1H), 2.320-2.485 (m, 2H), 1.960-2.180 (m, 3H), 1.502-1.848 (m, 9H), 1.495 (s, 3H), 1.336 (dq, 1H), 1.069-1.220 (m, 5H), 0.848 (s, 3H). ¹⁹F NMR (DMSO-d₆): −164.908. LCMS: 98%, MH⁺ 582.4 (exact mass 581.4 calcd for C₃₃H₄₀FNO₅S).

### Example 50

### 16,17-[(Cyclohexylmethylene)bis(oxy)]-11-hydroxy-21-methylthio-pregna-1,4-diene-3,20-dione[11β,16α(R)]

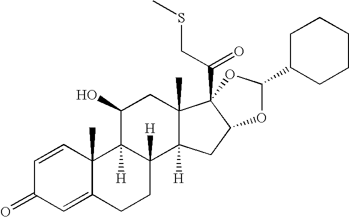

The mesylate described in Example 24 (1 equivalent) and the catalytic (0.2 equivalent) of sodium iodide were suspended in anhydrous acetonitrile (5 mL/mmol) and then solid sodium thiomethoxide (1.1 equivalent) was added with vigorous stirring at room temperature. The reaction mixture was occasionally analyzed by TLC (ethyl acetate/hexane 1:1) and after 48 hours the solvent was evaporated, the residue partitioned between dichloromethane and water and the separated organic layer was washed twice with saturated sodium bicarbonate solution, brine and dried over anhydrous magnesium sulfate. The crude product obtained after decantation and evaporation of the organic layer was purified by silica-gel chromatography eluting with the mixture of ethyl acetate/hexane (1:2).

### Example 51

### 16,17-[(Cyclohexylmethylene)bis(oxy)]-9-fluoro-11-hydroxy-21-methylthio-pregna-1,4-diene-3,20-dione[11β,16α(R)]

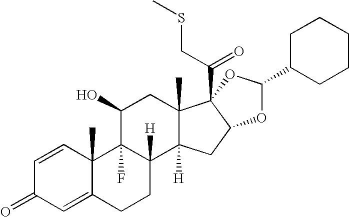

The title compound can prepared as described in Example 50, using the mesylate described in Example 25 as a starting material.

### Example 52

### 16,17-[(Tetrahydro-thiopyran-4-yl)bis(oxy)]-11,21-dihydroxypregna-1,4-diene-3,20-dione[11β,16α(R)]

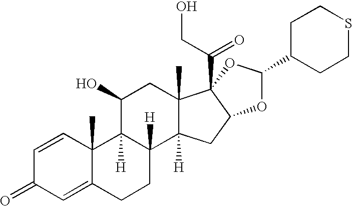

The title compound can be prepared analogously as described in Example 22, replacing cyclohexanecarboxaldehyde with tetrahydrothiopyran-4-yl-carboxaldehyde.

### Example 53

### 16,17-[(Tetrahydro-thiopyran-4-yl)bis(oxy)]-9-fluoro-11,21-dihydroxypregna-1,4-diene-3,20-dione[11β,16α(R)]

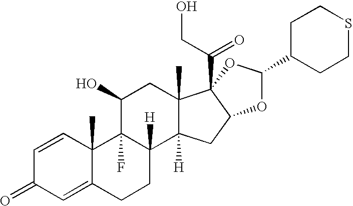

The title compound can be prepared analogously as described in Example 23, replacing cyclohexanecarboxaldehyde with tetrahydrothiopyran-4-yl-carboxaldehyde.

### Example 54

### 16,17-[(Tetrahydro-thiopyran-4-ylmethyl)bis(oxy)]-11,21-dihydroxypregna-1,4-diene-3,20-dione[11β,16α]

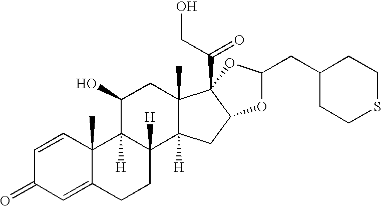

The title compound can be prepared analogously as described in Example 22, replacing cyclohexane-carboxaldehyde with tetrahydrothiopyran-4-yl-acetaldehyde.

### Example 55

### 16,17-[(Tetrahydro-thiopyran-4-ylmethyl)bis(oxy)]-9-fluoro-11,21-dihydroxypregna-1,4-diene-3,20-dione[11β,16α]

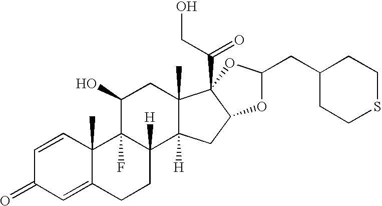

The title compound can be prepared analogously as described in Example 23, replacing cyclohexane-carboxaldehyde with tetrahydrothiopyran-4-yl-acetaldehyde.

Examples 56-103 describe the synthesis of steroid analogs according to Scheme V.

### Example 56

### 16,17-[(1-Methylpiperidyl-4-methylene)bis(oxy)]-11,21-dihydroxypregna-1,4-diene-3,20-dione[11β,16α]

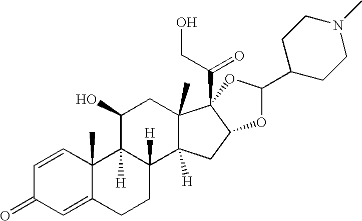

Desonide (1 equiv) was dissolved in 1-nitromethane (at concentration ca. 0.7M), then 1-methylpiperidine-4-carboxaldehyde (1.2 equiv), prepared according to Gray (1988), was added with stirring, followed by dropwise addition of 70% perchloric acid (4 equiv) at room temperature. The reaction mixture was stirred for 48 hours at room temperature and then worked-up as described in Example 22. The crude material was purified by silica gel chromatography using increasing amount (up to 10%) of methanol in chloroform. The title product was obtained as mixture of 22-epimers. LCMS: 56:43, both MH⁺ 486.4 (exact mass 485.4 calcd for C₂₈H₃₉NO₆).

### Example 57

### 16,17-[(1-Methylpiperidyl-4-methylene)bis(oxy)]-9-fluoro-11,21-dihydroxypregna-1,4-diene-3,20-dione[11β,16α]

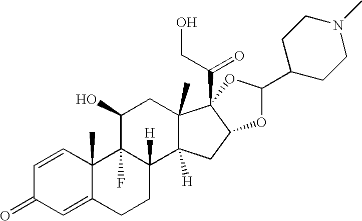

The title compound was synthesized as described in Example 56, substituting desonide with triamcinolone acetonide. ¹⁹F NMR (CDCl₃): −164.385 ppm (dd), 165.148 ppm (dd). LCMS: 45:50, both MH⁺ 504.4 (exact mass 503.4 calcd for C₂₈H₃₈FNO₆).

### Example 58

### 16,17-[Pyridinyl-4-methylene)bis(oxy)]-11,21-dihydroxypregna-1,4-diene-3,20-dione[11β,16α]

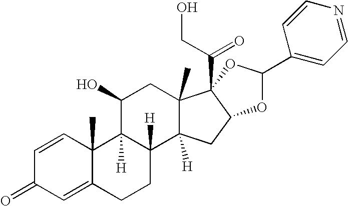

The title compound was prepared similarly as described in Example 56, except that 1-methyl-4-formylpiperidine was replaced by 4-pyridylcarboxaldehyde and additionally the reaction mixture was heated at 80° C. for 30 minutes. The crude product was purified by silica gel chromatography (0-10% of isopropanol in dichloromethane).

### Example 59

### 16,17-[Pyridinyl-3-methylene)bis(oxy)]-11,21-dihydroxypregna-1,4-diene-3,20-dione[11β,16α]

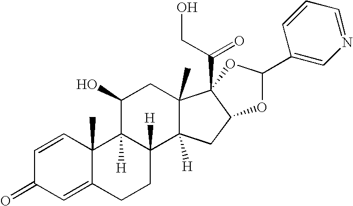

The title compound was prepared analogously as described in Example 58, substituting 4-pyridylcarboxaldehyde with 3-pyridylcarboxaldehyde. Final purification was accomplished by the preparative HPLC, yielding the title compound as monotrifluoroacetate.

¹H NMR (CDCl₃) indicated the presence of both 22-epimers in almost 1:1 ratio. LCMS: 98% (epimers not resolved) MH⁺ 466.3 (exact mass 465.2 calcd for C₂₇H₃₁NO₆).

### Example 60

### 16,17-[Pyridinyl-2-methylene)bis(oxy)]-11,21-dihydroxypregna-1,4-diene-3,20-dione[11β,16α]

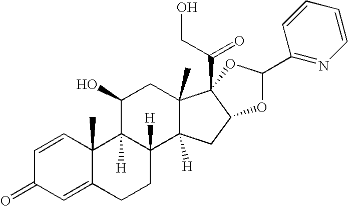

The title compound can be prepared analogously as described in Example 58, substituting 4-pyridylcarboxaldehyde with 2-pyridylcarboxaldehyde.

### Example 61

### 16,17-[Pyridinyl-4-methylene)bis(oxy)]-9-fluoro-11,21-dihydroxypregna-1,4-diene-3,20-dione[11β,16α]

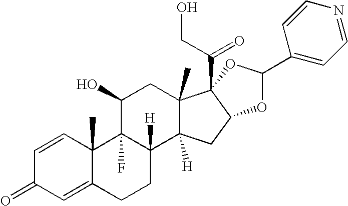

The title compound was prepared analogously as described in Example 58, substituting desonide with triamcinolone acetonide.

### Example 62

### 16,17-[Pyridinyl-3-methylene)bis(oxy)]-9-fluoro-11,21-dihydroxypregna-1,4-diene-3,20-dione[11β,16α]

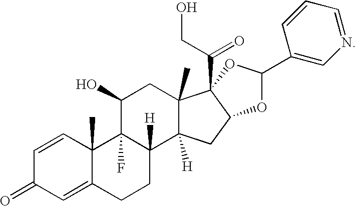

The steroid analog 14 was prepared analogously as described in Example 59, substituting desonide with triamcinolone acetonide. The crude product was purified by silica gel chromatography eluting with the increasing gradient of 2-propanol (0-10%) in dichloromethane, resolving 22-epimers (as well as the more polar regioisomer). The material obtained after evaporation of the separated fractions was recrystallized from a dichloromethane/diethyl ether mixture.

Analytical data for the 22-R epimer (confirmed by the 2D NMR study)-¹H NMR (DMSO-d₆): 8.604-8.642 (m, 2H), 7.810 (dt, 1H), 7.460 (dd, 1H), 7.282 (d, 1H), 6.230 (dd, 1H), 6.031 (bs, 1H), 5.603 (s, 1H), 5.463 (AB, 1H), 5.131 (dd, 1H), 4.979 (d, 1H), 4.536-4.601 (m, 1H), 4.152-4.245 (m, 2H), 2.510-2.667 (m, 2H), 2.363 (dd, 1H), 2.025-2.176 (m, 2H), 1.836-1.870 (m, 1H), 1.680-1.720 (m, 2H), 1.496 (s, 3H), 1.382 (dq, 1H), 1.235-1.260 (m, 1H), 0.880 (s, 3H). ¹⁹F NMR (DMSO-d₆): −165.463 ppm (dd, 1F). LCMS: 99%, MH⁺ 484.4 (exact mass 483.3 calcd for C₂₇H₃₀FNO₆). Anal. Calc: C, 67.07; H, 6.25; N, 2.90. Found: C, 66.90; H, 6.28; N, 2.92.

### Example 63

### 16,17-[Pyridinyl-2-methylene)bis(oxy)]-9-fluoro-11,21-dihydroxypregna-1,4-diene-3,20-dione[11β,16α]

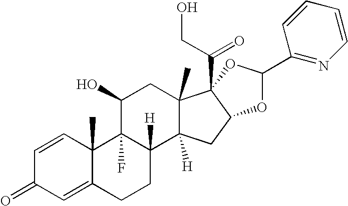

The title compound can be prepared analogously as described in Example 60, substituting desonide with triamcinolone acetonide.

### Examples 64

### 16,17-[2-Methoxy-pyridinyl-3-methylene)bis(oxy)]-11,21-dihydroxypregna-1,4-diene-3,20-dione[11β,16α]

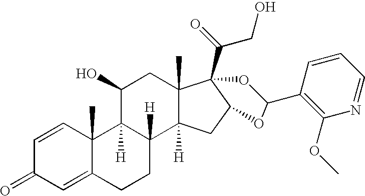

The title compound can be prepared as described in Example 58, substituting 4-pyridyl-carboxaldehyde with 2-methoxy-3-pirydyl-carboxaldehyde.

### Example 65

### 16,17-[2-Methoxy-pyridinyl-3-methylene)bis(oxy)]-9-fluoro-11,21-dihydroxypregna-1,4-diene-3,20-dione[11β,16α]

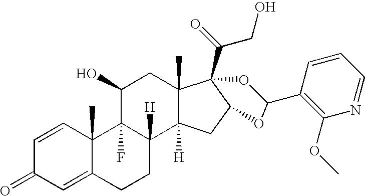

The title compound can be prepared as described in Example 59, substituting 4-pyridyl-carboxaldehyde with 2-methoxy-3-pirydyl-carboxaldehyde.

### Example 66

### 16,17-[2-Bromo-pyridinyl-3-methylene)bis(oxy)]-11,21-dihydroxypregna-1,4-diene-3,20-dione[11β,16α]

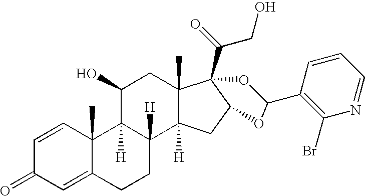

The title compound can be prepared as described in Example 58, substituting 4-pyridyl-carboxaldehyde with 2-bromo-3-pirydyl-carboxaldehyde.

### Example 67

### 16,17-[2-Bromo-pyridinyl-3-methylene)bis(oxy)]-9-fluoro-11,21-dihydroxypregna-1,4-diene-3,20-dione[11β,16α]

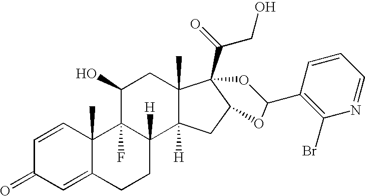

The title compound can be prepared as described in Example 59, substituting 4-pyridyl-carboxaldehyde with 2-methoxy-3-pirydyl-carboxaldehyde.

### Example 68

### 16,17-[6-Methoxy-pyridinyl-3-methylene)bis(oxy)]-11,21-dihydroxypregna-1,4-diene-3,20-dione[11β,16α]

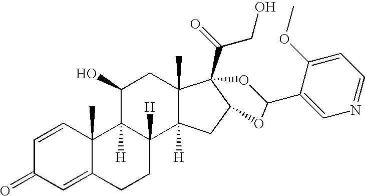

The title compound can be prepared as described in Example 58, substituting 4-pyridyl-carboxaldehyde with 6-methoxy-3-pirydyl-carboxaldehyde.

### Example 69

### 16,17-[6-Methoxy-pyridinyl-3-methylene)bis(oxy)]-9-fluoro-11,21-dihydroxypregna-1,4-diene-3,20-dione[11β,16α]

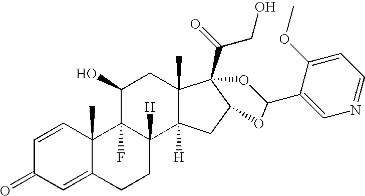

The title compound can be prepared as described in Example 59, substituting 4-pyridyl-carboxaldehyde with 6-methoxy-3-pirydyl-carboxaldehyde.

### Example 70

### 16,17-[3-Bromo-pyridinyl-4-methylene)bis(oxy)]-11,21-dihydroxypregna-1,4-diene-3,20-dione[11β,16α]

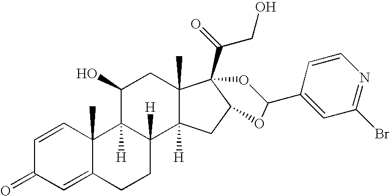

The title compound can be prepared as described in Example 58, substituting 4-pyridyl-carboxaldehyde with 3-bromo-4-pirydyl-carboxaldehyde.

### Example 71

### 16,17-[3-Bromo-pyridinyl-4-methylene)bis(oxy)]-9-fluoro-11,21-dihydroxypregna-1,4-diene-3,20-dione[11β,16α]

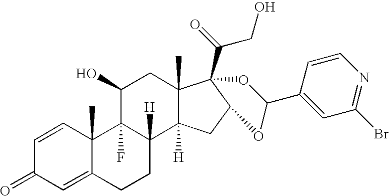

The title compound can be prepared as described in Example 59, substituting 4-pyridyl-carboxaldehyde with 3-bromo-4-pirydyl-carboxaldehyde.

### Example 72

### 16,17-[3-Chloro-pyridinyl-4-methylene)bis(oxy)]-11,21-dihydroxypregna-1,4-diene-3,20-dione[11β,16α]

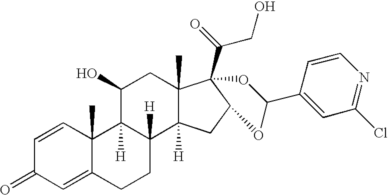

The title compound can be prepared as described in Example 58, substituting 4-pyridyl-carboxaldehyde with 3-chloro4-pirydyl-carboxaldehyde.

### Example 73

### 16,17-[3-Chloro-pyridinyl-4-methylene)bis(oxy)]-9-fluoro-11,21-dihydroxypregna-1,4-diene-3,20-dione[11β,16α]

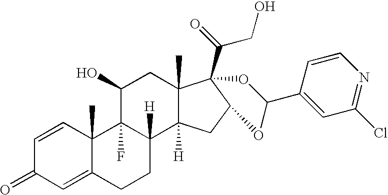

The title compound can be prepared as described in Example 59, substituting 4-pyridyl-carboxaldehyde with 3-chloro-4-pirydyl-carboxaldehyde.

### Example 74

### 16,17-[3-Fluoro-pyridinyl-4-methylene)bis(oxy)]-11,21-dihydroxypregna-1,4-diene-3,20-dione[11β,16α]

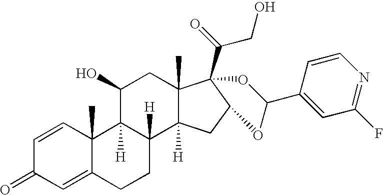

The title compound can be prepared as described in Example 58, substituting 4-pyridyl-carboxaldehyde with 3-fluoro-4-pirydyl-carboxaldehyde.

### Example 75

### 16,17-[3-Fluoro-pyridinyl-4-methylene)bis(oxy)]-9-fluoro-11,21-dihydroxypregna-1,4-diene-3,20-dione[11β,16α]

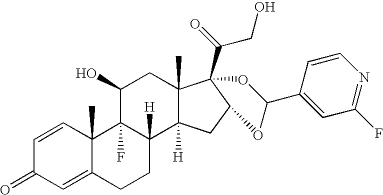

The title compound can be prepared as described in Example 59, substituting 4-pyridyl-carboxaldehyde with 3-fluoro-4-pirydyl-carboxaldehyde.

### Example 76

### 16,17-[8-Quinoline-3-yl-4-methylene)bis(oxy)]-11,21-dihydroxypregna-1,4-diene-3,20-dione[11β,16α]

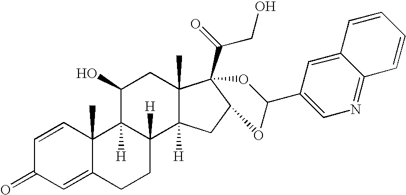

The title compound can be prepared as described in Example 58, substituting 4-pyridyl-carboxaldehyde with 8-quinoline-3-carboxaldehyde.

### Example 77

### 16,17-[8-Quinoline-3-yl-4-methylene)bis(oxy)]-9-fluoro-11,21-dihydroxypregna-1,4-diene-3,20-dione[11β,16α]

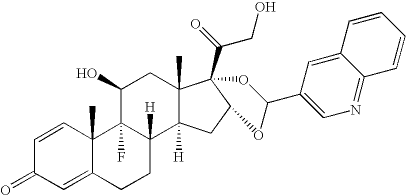

The title compound can be prepared as described in Example 59, substituting 4-pyridyl-carboxaldehyde with 8-quinoline-3-carboxaldehyde.

### Example 78

### 16,17-[8-Quinoline-4-yl-4-methylene)bis(oxy)]-11,21-dihydroxypregna-1,4-diene-3,20-dione[11β,16α]

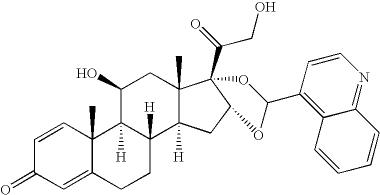

The title compound can be prepared as described in Example 58, substituting 4-pyridyl-carboxaldehyde with 8-quinoline-4-carboxaldehyde.

### Example 79

### 16,17-[8-Quinoline-4-yl-4-methylene)bis(oxy)]-9-fluoro-11,21-dihydroxypregna-1,4-diene-3,20-dione[11β,16α]

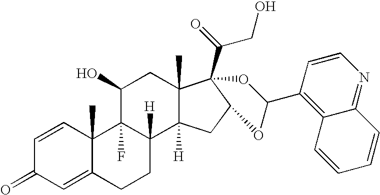

The title compound can be prepared as described in Example 59, substituting 4-pyridyl-carboxaldehyde with 8-quinoline-4-carboxaldehyde.

### Example 80

### 16,17-[8-Quinoline-2-yl-4-methylene)bis(oxy)]-11,21-dihydroxypregna-1,4-diene-3,20-dione[11β,16α]

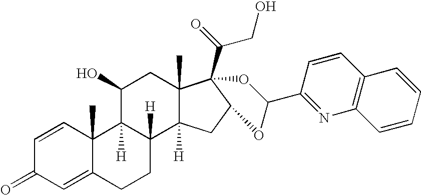

The title compound can be prepared as described in Example 58, substituting 4-pyridyl-carboxaldehyde with 8-quinoline-2-carboxaldehyde.

### Example 81

### 16,17-[8-Quinoline-2-yl-4-methylene)bis(oxy)]-9-fluoro-11,21-dihydroxypregna-1,4-diene-3,20-dione[11β,16α]

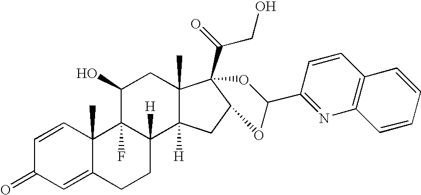

The title compound can be prepared as described in Example 59, substituting 4-pyridyl-carboxaldehyde with 8-quinoline-2-carboxaldehyde.

### Example 82

### 16,17-[Pyridinyl-3-methylene)bis(oxy)]-11-hydroxy-21-cyano-pregna-1,4-diene-3,20-dione[11β,16α]

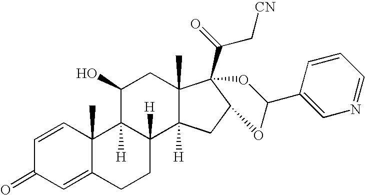

The title compound was prepared by the following two-step procedure. The steroid analog described in Example 59 was converted to the 21-mesylate derivative applying the procedure described in Example 24. The dry crystalline intermediate thus obtained was suspended in anhydrous acetonitrile (5 mL/mmol), followed by addition of excess of tetraethylammonium cyanide (2.2 equivalents) and the catalytic (0.2 equivalent) amount of sodium iodide. The LCMS analysis after stirring overnight at room temperature revealed the complete consumption of the mesylate and the formation of the 22-epimers of the desired product next to the pair of regioisomers (the 20-cyano-20,21-epoxy steroids are formed). The reaction mixture was then heated at 90° C. for 30 minutes leading to the ultimate clean formation of the desired β-cyano-ketosteroid. The workup consisted of dilution with ethyl acetate, followed by washing with saturated sodium bicarbonate (twice), brine and drying over anhydrous magnesium sulfate. The crude product was purified by recrystallization from dichloromethane/diethyl ether.

### Example 83

### 16,17-[Pyridinyl-3-methylene)bis(oxy)]-9-fluoro-11-hydroxy-21-cyano-pregna-1,4-diene-3,20-dione[11β,16α]

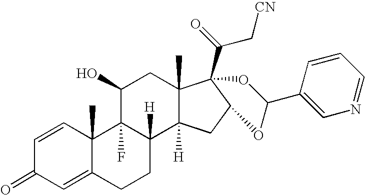

The title steroid 15 was synthesized from the analog 14 (described in Example 62) applying the two-step procedure described in Example 82.

LCMS: 99% (sum of epimers), MH⁺ 493.2 (exact mass 492.2 calcd for C₂₈H₂₉FN₂O₅). Anal. Calc: C, 68.28; H, 5.93; N, 5.69. Found: C, 67.34; H, 5.87; N, 5.47.

### Example 84

### 16,17-[Pyridinyl-4-methylene)bis(oxy)]-11-hydroxy-21-cyano-pregna-1,4-diene-3,20-dione[11β,16α]

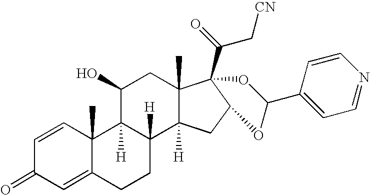

The title compound can be synthesized from the steroid described in Example 58 applying the two-step procedure described in Example 82.

### Example 85

### 16,17-[Pyridinyl-4-methylene)bis(oxy)]-9-fluoro-11-hydroxy-2′-cyano-pregna-1,4-diene-3,20-dione[11β,16α]

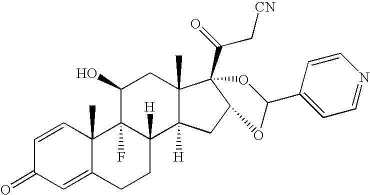

The title compound can be synthesized from the steroid described in Example 61 applying the two-step procedure described in Example 82.

### Example 86

### 16,17-[2-Methoxy-pyridinyl-3-methylene)bis(oxy)]-11-hydroxy-21-cyano-pregna-1,4-diene-3,20-dione[11β,16α]

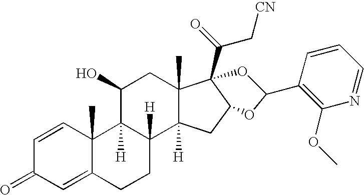

The title compound can be synthesized from the steroid described in Example 64 applying the two-step procedure described in Example 82.

### Example 87

### 16,17-[2-Methoxy-pyridinyl-3-methylene)bis(oxy)]-9-fluoro-11-hydroxy-21-cyano-pregna-1,4-diene-3,20-dione[11β,16α]

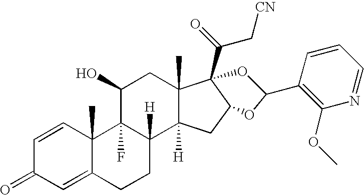

The title compound can be synthesized from the steroid described in Example 65 applying the two-step procedure described in Example 82.

### Example 88

### 16,17-[2-Bromo-pyridinyl-3-methylene)bis(oxy)]-11-hydroxy-21-cyano-pregna-1,4-diene-3,20-dione[11β,16α]

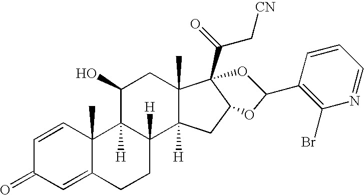

The title compound can be synthesized from the steroid described in Example 66 applying the two-step procedure described in Example 82.

### Example 89

### 16,17-[2-Bromo-pyridinyl-3-methylene)bis(oxy)]-9-fluoro-11-hydroxy-21-cyano-pregna-1,4-diene-3,20-dione[11β,16α]

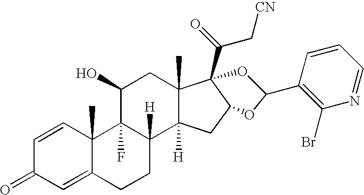

The title compound can be synthesized from the steroid described in Example 67 applying the two-step procedure described in Example 82.

### Example 90

### 16,17-[6-Methoxy-pyridinyl-3-methylene)bis(oxy)]-11-hydroxy-21-cyano-pregna-1,4-diene-3,20-dione[11β,16α]

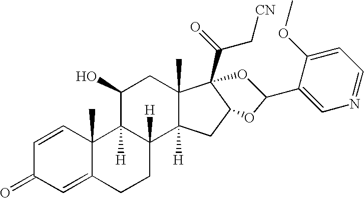

The title compound can be synthesized from the steroid described in Example 68 applying the two-step procedure described in Example 82.

### Example 91

### 16,17-[6-Methoxy-pyridinyl-3-methylene)bis(oxy)]-9-fluoro-11-hydroxy-21-cyano-pregna-1,4-diene-3,20-dione[11β,16α]

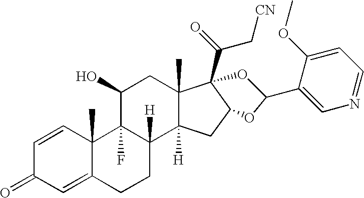

The title compound can be synthesized from the steroid described in Example 69 applying the two-step procedure described in Example 82.

### Example 92

### 16,17-[3-Bromo-pyridinyl-4-methylene)bis(oxy)]-11-hydroxy-21-cyano-pregna-1,4-diene-3,20-dione[11β,16α]

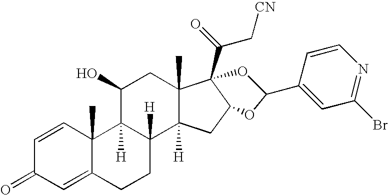

The title compound can be synthesized from the steroid described in Example 70 applying the two-step procedure described in Example 82.

### Example 93

### 16,17-[3-Bromo-pyridinyl-4-methylene)bis(oxy)]-9-fluoro-11-hydroxy-21-cyano-pregna-1,4-diene-3,20-dione[11β,16α]

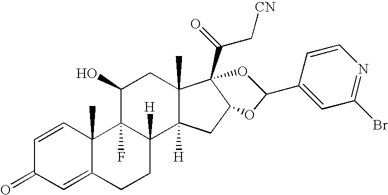

The title compound can be synthesized from the steroid described in Example 71 applying the two-step procedure described in Example 82.

### Example 94

### 16,17-[3-Chloro-pyridinyl-4-methylene)bis(oxy)]-11-hydroxy-21-cyano-pregna-1,4-diene-3,20-dione[11β,16α]

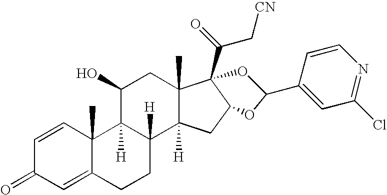

The title compound can be synthesized from the steroid described in Example 72 applying the two-step procedure described in Example 82.

### Example 95

### 16,17-[3-Chloro-pyridinyl-4-methylene)bis(oxy)]-9-fluoro-11-hydroxy-21-cyano-pregna-1,4-diene-3,20-dione[11β,16α]

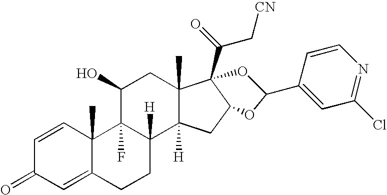

The title compound can be synthesized from the steroid described in Example 73 applying the two-step procedure described in Example 82.

### Example 96

### 16,17-[3-Fluoro-pyridinyl-4-methylene)bis(oxy)]-11-hydroxy-21-cyano-pregna-1,4-diene-3,20-dione[11β,16α]

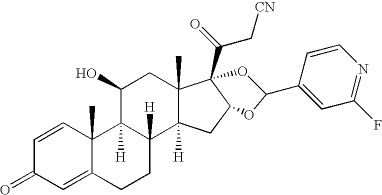

The title compound can be synthesized from the steroid described in Example 74 applying the two-step procedure described in Example 82.

### Example 97

### 16,17-[3-Fluoro-pyridinyl-4-methylene)bis(oxy)]-9-fluoro-11-hydroxy-21-cyano-pregna-1,4-diene-3,20-dione[11β,16α]

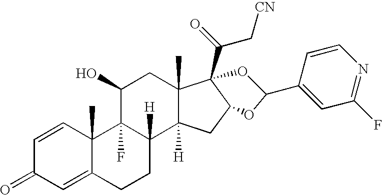

The title compound can be synthesized from the steroid described in Example 75 applying the two-step procedure described in Example 82.

### Example 98

### 16,17-[8-Quinoline-3-yl-4-methylene)bis(oxy)]-11-hydroxy-21-cyano-pregna-1,4-diene-3,20-dione[11β,16α]

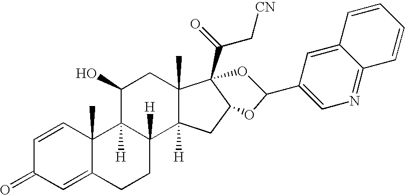

The title compound can be synthesized from the steroid described in Example 76 applying the two-step procedure described in Example 82.

### Example 99

### 16,17-[8-Quinoline-3-yl-4-methylene)bis(oxy)]-9-fluoro-11-hydroxy-21-cyano-pregna-1,4-diene-3,20-dione[11β,16α]

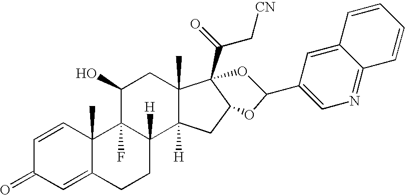

The title compound can be synthesized from the steroid described in Example 77 applying the two-step procedure described in Example 82.

### Example 100

### 16,17-[8-Quinoline-4-yl-4-methylene)bis(oxy)]-11-hydroxy-21-cyano-pregna-1,4-diene-3,20-dione[11β,16α]

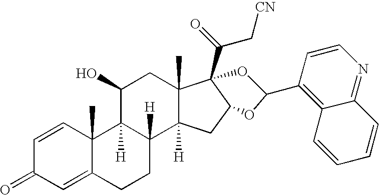

The title compound can be synthesized from the steroid described in Example 78 applying the two-step procedure described in Example 82.

### Example 101

### 16,17-[8-Quinoline-4-yl-4-methylene)bis(oxy)]-9-fluoro-11-hydroxy-21-cyano-pregna-1,4-diene-3,20-dione[11β,16α]

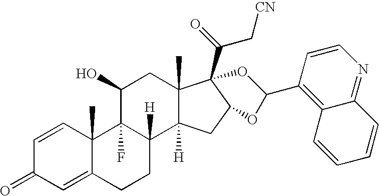

The title compound can be synthesized from the steroid described in Example 79 applying the two-step procedure described in Example 82.

### Example 102

### 16,17-[8-Quinoline-2-yl-4-methylene)bis(oxy)]-11-hydroxy-21-cyano-pregna-1,4-diene-3,20-dione[11β,16α]

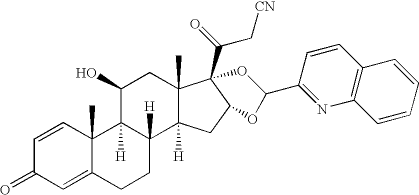

The title compound can be synthesized from the steroid described in Example 80 applying the two-step procedure described in Example 82.

### Example 103

### 16,17-[8-Quinoline-2-yl-4-methylene)bis(oxy)]-9-fluoro-11-hydroxy-21-cyano-pregna-1,4-diene-3,20-dione[11β,16α]

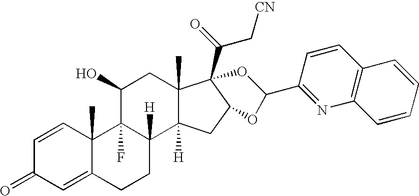

The title compound can be synthesized from the steroid described in Example 81 applying the two-step procedure described in Example 82.

Examples 104-117 illustrate the synthesis of the mutual prodrugs described on Scheme VI.

### Example 104

### N-Boc-Salmeterol-di-tert-butylphosphate-16,17-[(Cyclohexylmethylene)bis(oxy)]-11-hydroxy-21-(4-methylpiperazinium)-pregna-1,4-diene-3,20-dione[11β,16α(R)]

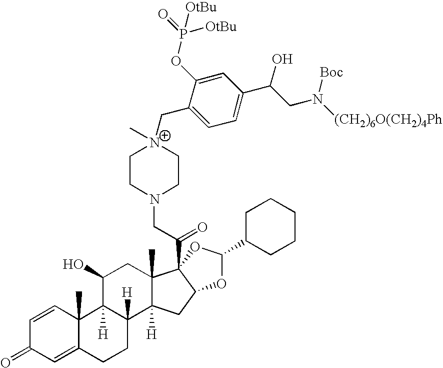

The 1.1 equivalent of mesylate 3 (described in Example 6), steroid analog described in Example 26 (1 equivalent) and sodium iodide (1 equivalent) were dissolved in a minimum amount of anhydrous acetonitrile with stirring at room temperature. The reaction mixture was monitored by TLC and LCMS. After 3 days the reaction mixture was concentrated and purified by silica gel chromatography using a mixture of dichloromethane/methanol/triethylamine (96:3:1). Fractions containing the desired quaternary ammonium salt were pooled, evaporated and the residue triturated with diethyl ether. Solids thus formed were filtered, washed with ether and dried.

LCMS: M+ 1243 (exact mass 1242.7 calcd for C₇₁H₁₀₉N₃O₁₃P⁺).

### Example 105

### N-Boc-Salmeterol-di-tert-butylphosphate-16,17-[(Cyclohexylmethylene)bis(oxy)]-9-fluoro-11-hydroxy-21-(4-methylpiperazinium)-pregna-1,4-diene-3,20-dione[11β,16α(R)]

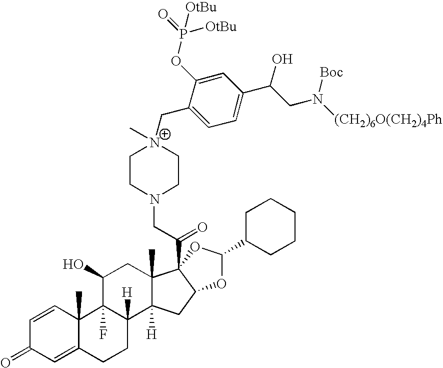

The title compound was prepared as described in Example 104, using the steroid 13 (described in Example 27) as a starting material.

LCMS: M+ 1261 (exact mass 1260.7 calcd for C₇₁H₁₀₈FN₃O₁₃P⁺).

### Example 106

### Salmeterol-phosphate-16,17-[(Cyclohexylmethylene)bis(oxy)]-11-hydroxy-21-(4-methylpiperazinium)-pregna-1,4-diene-3,20-dione[11β,16α(R)]

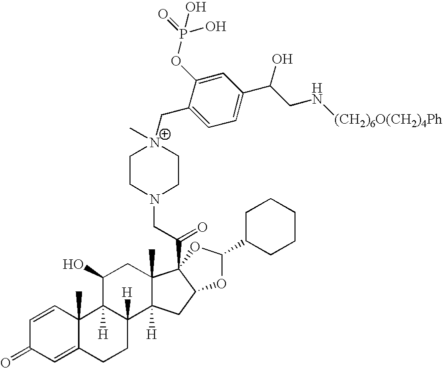

The quaternary ammonium salt described in Example 104 was treated with fresh, anhydrous 4N HCl in dioxane (2 mL) with stirring under nitrogen at room temperature. The progress of deprotection was monitored by TLC and LCMS. After 1 hour diethyl ether was added through septum and stirring was continued for another hour. Then the precipitate formed was filtered-off, washed thoroughly with ether, dried and recrystallized from mixture of dichloromethane/diethyl ether (yielding a dihydrochloride salt). If necessary, further purification can be achieved by chromatography using Isolute-C18 (Biotage) eluting with the increasing gradient of acetonitrile in water with 1% acetic acid (yielding the diacetate salt).

³¹PNMR (DMSO-d₆): −5.718 ppm. LCMS: 95%, M⁺ 1030.5 (exact mass 1030.59 calcd for C₅₈H₈₆Cl₂N₃O₁₁P⁺).

### Example 107

### Salmeterol-phosphate-16,17-[(Cyclohexylmethylene)bis(oxy)]-9-fluoro-11-hydroxy-21-(4-methylpiperazinium)-pregna-1,4-diene-3,20-dione[11β,16α(R)]

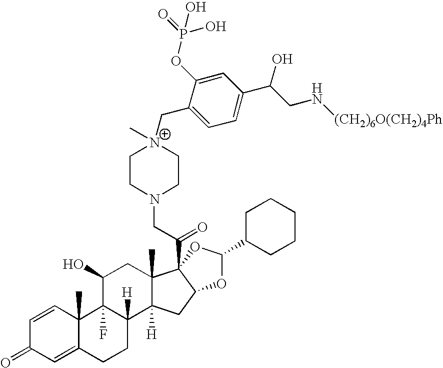

The mutual prodrug 16 was prepared as described in Example 106, using the quaternary ammonium salt described in Example 105 as a starting material.

³¹P NMR (DMSO-d₆): −6.018 ppm. ¹⁹F NMR (DMSO-d₆): −165.361 ppm (dd, J=8 Hz, J=32 Hz). LCMS: 96%, M+ 1049.3 (exact mass 1049.2 calcd for C₅₈H₈₄FN₃O₁₁P⁺).

### Example 108

### N-Boc-Albuterol-di-tert-butylphosphate-16,17-[(Cyclohexylmethylene)bis(oxy)]-9-fluoro-11-hydroxy-21-(4-methylpiperazinium)-pregna-1,4-diene-3,20-dione[11β,16α(R)]

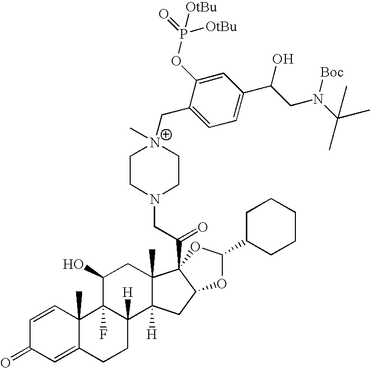

The title compound can be prepared according to the procedure described in Example 104, using the mesylate 7 (see Example 13) and the steroid 13 (see Example 27) as the starting materials.

### Example 109

### Albuterol-phosphate-16,17-[(Cyclohexylmethylene)bis(oxy)]-9-fluoro-11-hydroxy-21-(4-methylpiperazinium)-pregna-1,4-diene-3,20-dione[11β,16α(R)]

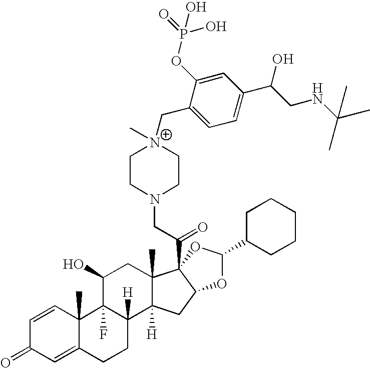

The title mutual prodrug can be prepared from the quaternary ammonium salt described in Example 108 by the procedure described in Example 106.

### Example 110

### N-Boc-Salmeterol-di-tert-butylphosphate-16,17-[(Cyclohexylmethylene)bis(oxy)]-9-fluoro-11-hydroxy-21-(imidazolium)-pregna-1,4-diene-3,20-dione[11β,16α(R)]

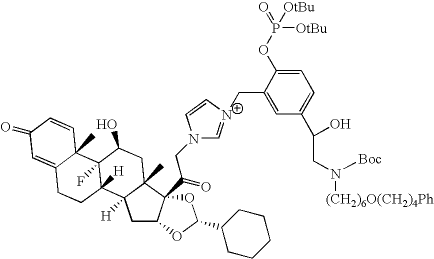

The title compound can be prepared according to the procedure described in Example 104, using the mesylate 3 (see Example 6) and the steroid described in Example 45 as the starting materials.

### Example 111

### Salmeterol-phosphate-16,17-[(Cyclohexylmethylene)bis(oxy)]-9-fluoro-11-hydroxy-21-(imidazolium)-pregna-1,4-diene-3,20-dione[11β,16α(R)]

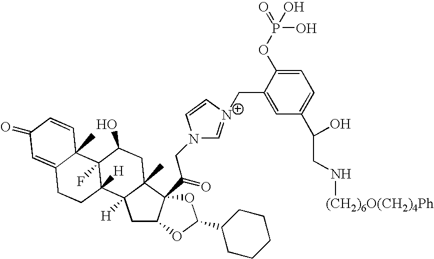

The title mutual prodrug can be prepared from the quaternary imidazolium salt described in Example 110 by the procedure described in Example 106.

### Example 112

### N-Boc-Albuterol-di-tert-butylphosphate-16,17-[(Cyclohexylmethylene)bis(oxy)]-9-fluoro-11-hydroxy-21-(imidazolium)-pregna-1,4-diene-3,20-dione[11β,16α(R)]

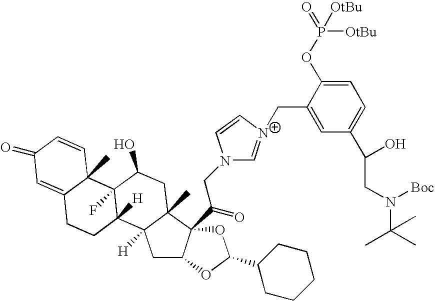

The title compound can be prepared according to the procedure described in Example 104, using the mesylate 7 (see Example 13) and the steroid described in Example 45 as the starting materials.

### Example 113

### Albuterol-phosphate-16,17-[(Cyclohexylmethylene)bis(oxy)]-9-fluoro-11-hydroxy-21-(imidazolium)-pregna-1,4-diene-3,20-dione[11β,16α(R)]

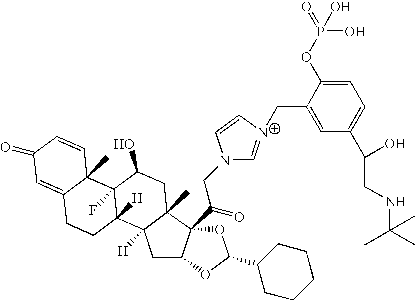

The title mutual prodrug can be prepared from the quaternary imidazolium salt described in Example 112 according to the procedure described in example 106.

### Example 114

### N-Boc-Salmeterol-di-tert-butylphosphate-16,17-[(Cyclohexylmethylene)bis(oxy)]-9-fluoro-11-hydroxy-21-methylsulfonium-pregna-1,4-diene-3,20-dione[11β,16α(R)]

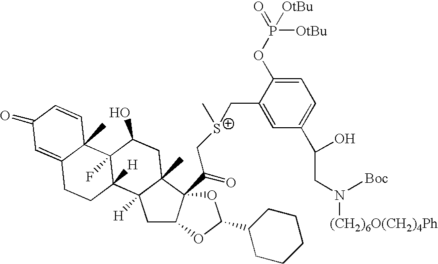

The title compound can be prepared according to the procedure described in Example 104, using the mesylate 3 (see Example 6) and the steroid described in Example 51 as the starting materials.

### Example 115

### Salmeterol-phosphate-16,17-[(Cyclohexylmethylene)bis(oxy)]-9-fluoro-11-hydroxy-21-methylsulfonium-pregna-1,4-diene-3,20-dione[11β,16α(R)]

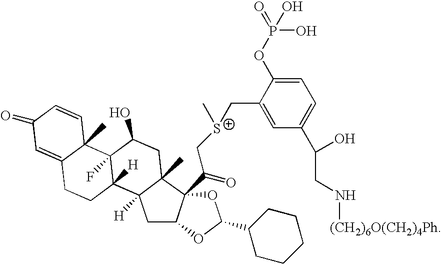

The title mutual prodrug can be prepared from the compound described in Example 114 according to the procedure described in Example 106.

### Example 116

### N-Boc-Albuterol-di-tert-butylphosphate-16,17-[(Cyclohexylmethylene)bis(oxy)]-9-fluoro-11-hydroxy-21-methylsulfonium-pregna-1,4-diene-3,20-dione[11β,16α(R)]

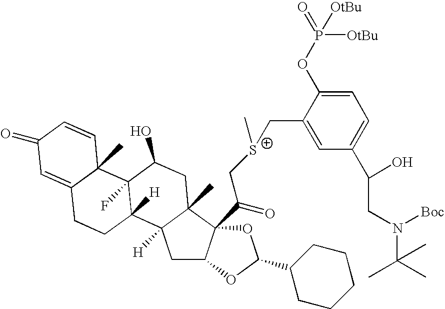

The title compound can be prepared according to the procedure described in Example 104, using the mesylate 7 (see Example 13) and the steroid described in Example 51 as the starting materials.

### Example 117

### Albuterol-phosphate-16,17-[(Cyclohexylmethylene)bis(oxy)]-9-fluoro-11-hydroxy-21-methylsulfonium-pregna-1,4-diene-3,20-dione[11β,16α(R)]

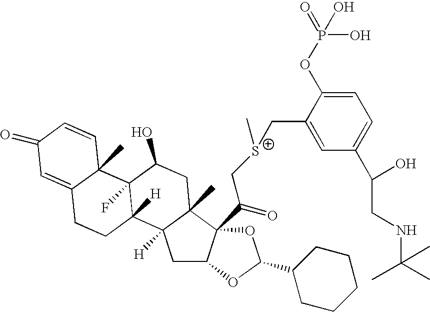

The title mutual prodrug can be prepared from the compound described in Example 116 according to the procedure described in Example 106.

Examples 118-139 illustrate synthesis of mutual prodrugs according to Scheme VII.

### Example 118

### 16,17-[(Tetrahydro-thiopyran-4-yl)bis(oxy)]-9-fluoro-11-hydroxy-21-trityloxy-pregna-1,4-diene-3,20-dione[11β,16α(R)]

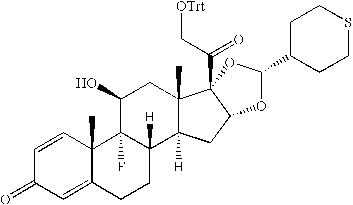

Steroid described in Example 53 (1 equivalent) and DMAP (0.1 equivalent) was dissolved in anhydrous dichloromethane (5 mL/mmol), which was followed by the dropwise addition of triethylamine (2 equivalents) followed by solid triphenylmethyl chloride (2 equivalents) in portions with vigorous stirring while cooling the reaction mixture in a water bath. The TLC analysis after overnight reaction showed consumption of almost all starting steroid. The mixture was quenched with a few drops of methanol, diluted with dichloromethane and washed with 10% citric acid, saturated sodium bicarbonate and finally brine. After drying of the organic layer over anhydrous magnesium sulfate, decantation and evaporation the crude product was purified by silica gel chromatography using the increasing amount of ethyl acetate in hexane (1:3 to 1:1).

### Example 119

### N-Boc-Salmeterol-di-tert-butylphosphate-16,17-[(Tetrahydro-thiopyranylium)bis(oxy)]-9-fluoro-11-hydroxy-21-trityloxy-pregna-1,4-diene-3,20-dione[11β,16α(R)]

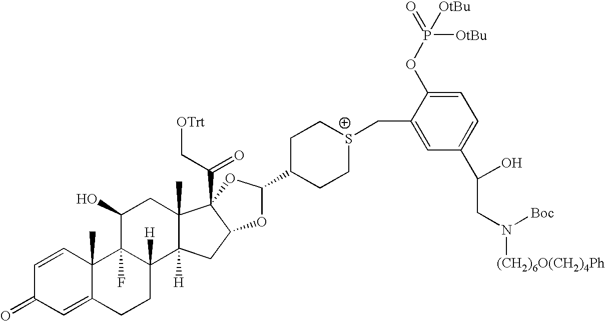

The title compound can be prepared according to the procedure described in Example 104, using the mesylate 3 (see Example 6) and the steroid described in Example 118 as starting materials.

### Example 120

### Salmeterol-phosphate-16,17-[(Tetrahydro-thiopyranylium)bis(oxy)]-9-fluoro-11,21-dihydroxy-pregna-1,4-diene-3,20-dione[11β,16α(R)]

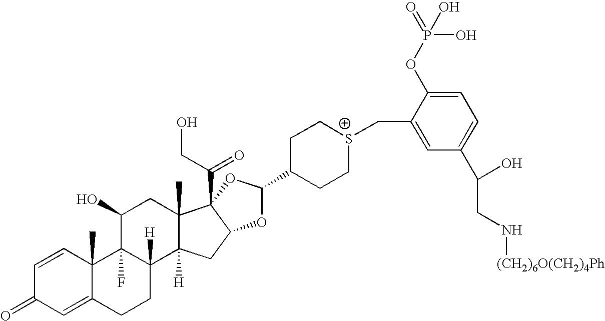

The title mutual prodrug can be prepared from the sulfonium salt described in Example 119 according to the procedure described in Example 106.

### Example 121

### N-Boc-Albuterol-di-tert-butylphosphate-16,17-[(Tetrahydro-thiopyranylium)bis(oxy)]-9-fluoro-11-hydroxy-21-trityloxy-pregna-1,4-diene-3,20-dione[11β,16α(R)]

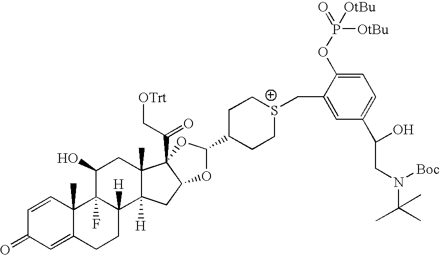

The title compound can be prepared according to the procedure described in Example 104, using the mesylate 7 (see Example 13) and the steroid described in Example 118 as starting materials.

### Example 122

### Albuterol-phosphate-16,17-[(Tetrahydro-thiopyranylium)bis(oxy)]-9-fluoro-11,21-dihydroxy-pregna-1,4-diene-3,20-dione[11β,16α(R)]

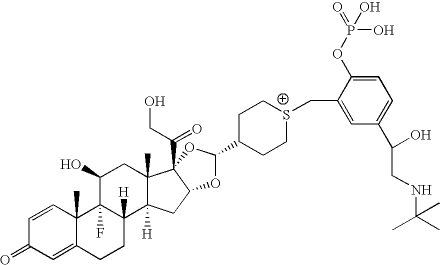

The title mutual prodrug can be prepared from the sulfonium salt described in Example 121 according to the procedure described in Example 106.

### Example 123

### 16,17-[(1-Methylpiperidyl-4-methylene)bis(oxy)]-11-hydroxy-21-trityloxy-pregna-1,4-diene-3,20-dione[11β,16α]

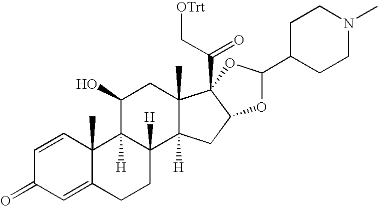

The title compound can be prepared from the steroid described in Example 56 using the procedure described in Example 118.

### Example 124

### N-Boc-Salmeterol-di-tert-butylphosphate-16,17-[(1-Methylpiperidinium-4-methylene)bis(oxy)]-11-hydroxy-21-trityloxy-pregna-1,4-diene-3,20-dione[11β,16α]

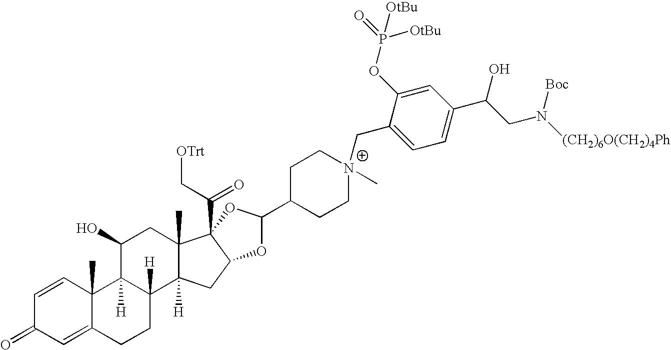

The title compound can be prepared according to the procedure described in Example 104, using the mesylate 3 (see Example 6) and the steroid described in Example 123 as starting materials.

### Example 125

### 16,17-[(1-Methylpiperidyl-4-methylene)bis(oxy)]-9-fluoro-11-hydroxy-21-trityloxy-pregna-1,4-diene-3,20-dione[11β,16α]

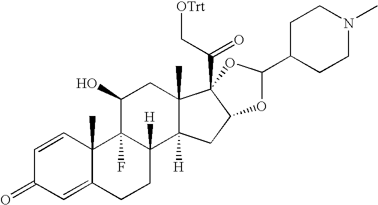

The title compound can be synthesized from the steroid described in Example 57 according to the procedure described in Example 118.

### Example 126

### N-Boc-Salmeterol-di-tert-butylphosphate-16,17-[(1-Methylpiperidinium-4-methylene)bis(oxy)]-9-fluoro-11-hydroxy-21-trityloxy-pregna-1,4-diene-3,20-dione[11β,16α]

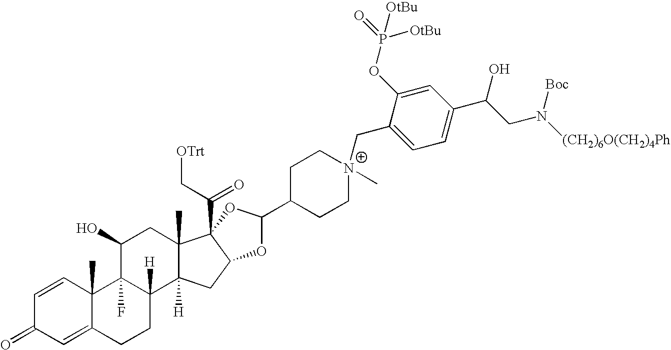

The title compound can be prepared according to the procedure described in Example 104, using the mesylate 3 (see Example 6) and the steroid described in Example 125 as starting materials.

### Example 127

### Salmeterol-phosphate-16,17-[(1-Methylpiperidinium-4-methylene)bis(oxy)]-11,21-dihydroxypregna-1,4-diene-3,20-dione[11β,16α]

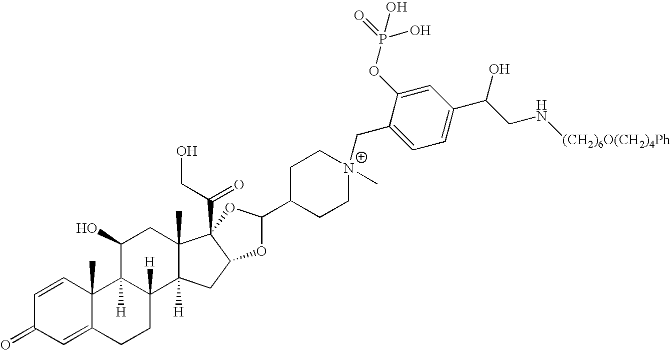

The title compound can be prepared according to the procedure described in Example 106 using the quaternary ammonium salt described in Example 124.

### Example 128

### Salmeterol-phosphate-16,17-[(1-Methylpiperidinium-4-methylene)bis(oxy)]-9-fluoro-11,21-dihydroxypregna-1,4-diene-3,20-dione[11β,16α]

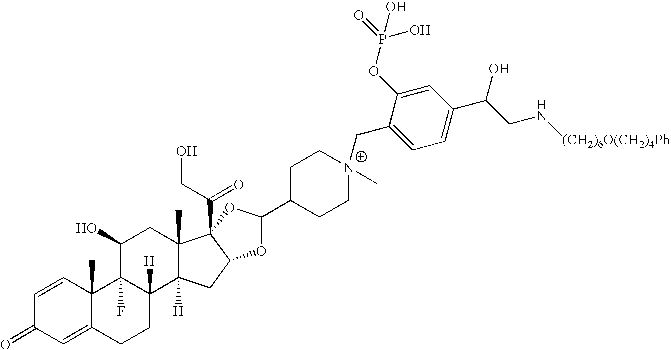

The title compound can be prepared according to the procedure described in Example 106 using the quaternary ammonium salt described in Example 126.

### Example 129

### N-Boc-Albuterol-di-tert-butylphosphate-16,17-[(1-Methylpiperidinium-4-methylene)bis(oxy)]-9-fluoro-11-hydroxy-21-trityloxy-pregna-1,4-diene-3,20-dione[11β,16α]

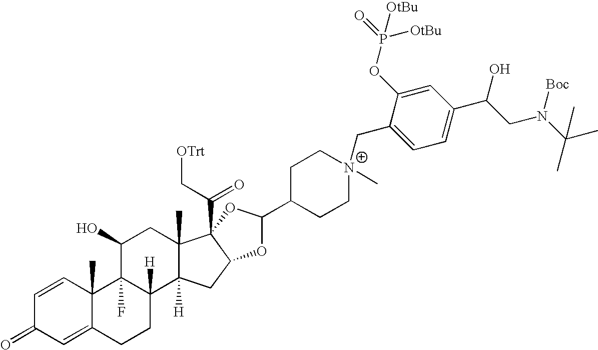

The title compound can be prepared according to the procedure described in Example 104, using the mesylate 7 (see Example 13) and the steroid described in Example 125 as starting materials.

### Example 130

### Albuterol-phosphate-16,17-[(1-Methylpiperidinium-4-methylene)bis(oxy)]-9-fluoro-11,21-hydroxy-pregna-1,4-diene-3,20-dione[11β,16α]

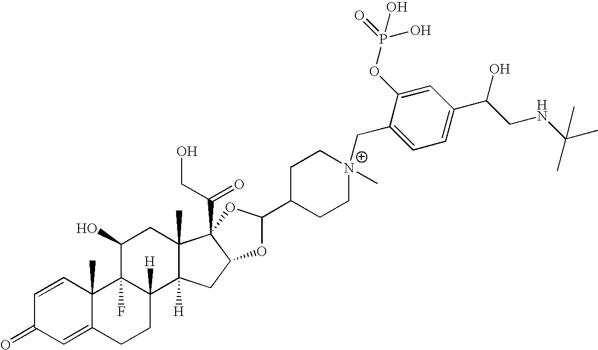

The title mutual prodrug can be prepared from the quaternary ammonium salt described in Example 129 according to the procedure described in Example 106

### Example 131

### 16,17-[Pyridinyl-3-methylene)bis(oxy)]-9-fluoro-11-hydroxy-21-trityloxy-pregna-1,4-diene-3,20-dione[11β,16α]

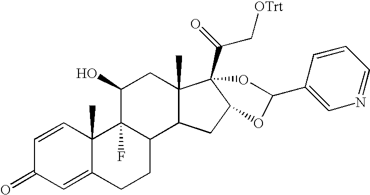

The title compound can be synthesized from the steroid 14 (described in Example 62) according to the procedure described in Example 118.

### Example 132

### N-Boc-Salmeterol-di-tert-butylphosphate-16,17-[Pyridynium-3-methylene)bis(oxy)]-9-fluoro-11-hydroxy-21-trityloxy-pregna-1,4-diene-3,20-dione[11β,16α]

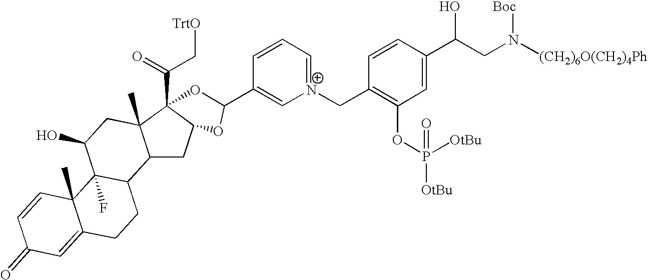

The title compound was prepared according to the procedure described in Example 104, using the mesylate 3 (see Example 6) and the steroid described in Example 131 as starting materials.

LCMS: M+ 1414.7 (exact mass 1415.7 calcd for C₈₄H₁₀₅FN₂O₁₄P⁺).

### Example 133

### Salmeterol-phosphate-16,17-[Pyridynium-3-methylene)bis(oxy)]-9-fluoro-11,21-dihydroxypregna-1,4-diene-3,20-dione[11β,16α]

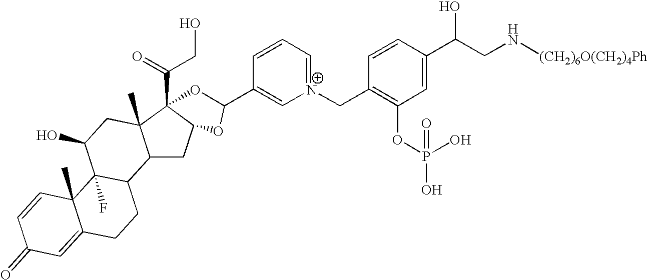

The mutual prodrug 17 was prepared according to the procedure described in Example 106 from the pyridinium salt described in Example 132 and purified by reverse phase chromatography using the Isolute-C18 column (Biotage) eluting with the increasing amount of acetonitrile (0-50%) in water acidified with 2% of acetic acid. After lyophilization obtained as the diacetate

³¹P NMR (DMSO-d₆): −4.116 ppm. ¹⁹F NMR (DMSO-d₆): −165.124-−164.480 ppm (multiplet). LCMS: 97% M+ 961.5 (exact mass 961.44 calcd for C₅₂H₆₇FN₂O₁₂P⁺). Anal. Calcd for C₅₆H₇₄FN₂O₁₆P % C, 62.21; % H, 6.90; % N, 2.59. Found % C, 62.13; % H, 6.85; % N, 2.76.

### Example 134

### N-Boc-Albuterol-di-tert-butylphosphate-16,17-[Pyridynium-3-methylene)bis(oxy)]-9-fluoro-11-hydroxy-21-trityloxy-pregna-1,4-diene-3,20-dione[11β,16α]

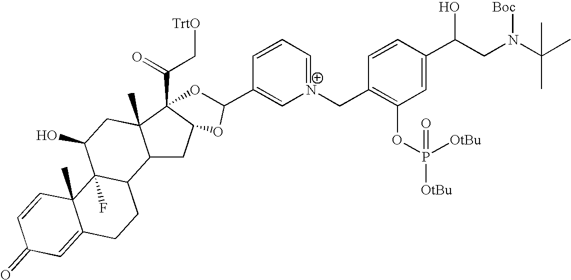

The title compound can be prepared according to the procedure described in Example 104, using the mesylate 7 (see Example 13) and the steroid described in Example 131 as starting materials.

### Example 135

### Albuterol-phosphate-16,17-[Pyridynium-3-methylene)bis(oxy)]-9-fluoro-11,21-dihydroxypregna-1,4-diene-3,20-dione[11β,16α]

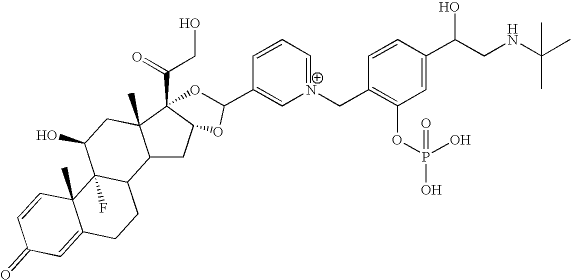

The title mutual prodrug can be prepared according to the Procedure described in Example 106 from the pyridinium salt described in Example 134.

### Example 136

### N-Boc-Salmeterol-di-tert-butylphosphate-16,17-[Pyridynium-3-methylene)bis(oxy)]-9-fluoro-11-hydroxy-21-cyano-pregna-1,4-diene-3,20-dione[11β,16α]

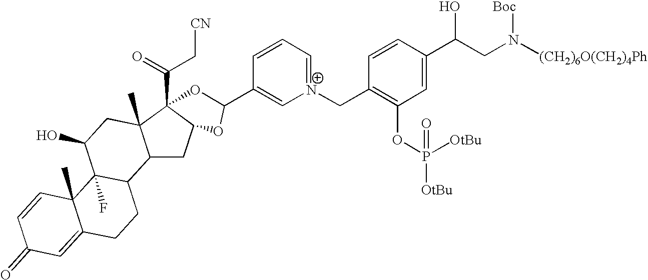

The title compound can be prepared according to the procedure described in Example 104, using the mesylate 3 (see Example 6) and the steroid 15 (described in Example 83) as starting materials.

### Example 137

### Salmeterol-phosphate-16,17-[Pyridynium-3-methylene)bis(oxy)]-9-fluoro-11-hydroxy-21-cyano-pregna-1,4-diene-3,20-dione[11β,16α]

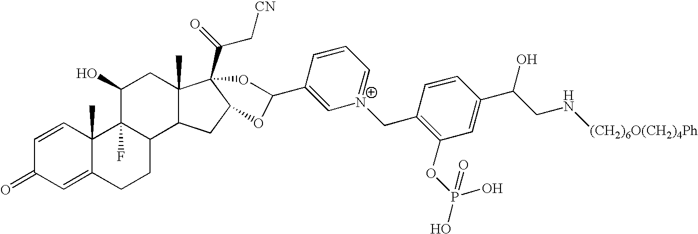

The title mutual prodrug can be prepared according to the procedure described in Example 106 starting from the pyridinium salt described in Example 136.

### Example 138

### N-Boc-Albuterol-di-tert-butylphosphate-16,17-[Pyridynium-3-methylene)bis(oxy)]-9-fluoro-11-hydroxy-21-cyano-pregna-1,4-diene-3,20-dione[11β,16α]

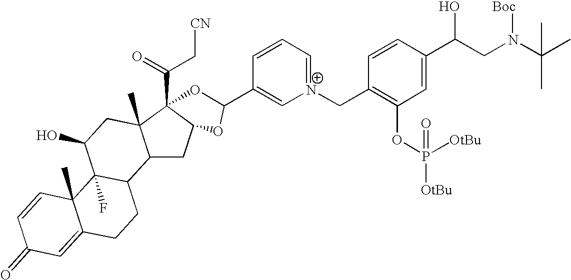

The title compound can be prepared according to the procedure described in Example 104, using the mesylate 7 (see Example 13) and the steroid 15 (described in Example 83) as starting materials.

### Example 139

### Albuterol-phosphate-16,17-[Pyridynium-3-methylene)bis(oxy)]-9-fluoro-11-hydroxy-21-cyano-pregna-1,4-diene-3,20-dione[11β,16α]

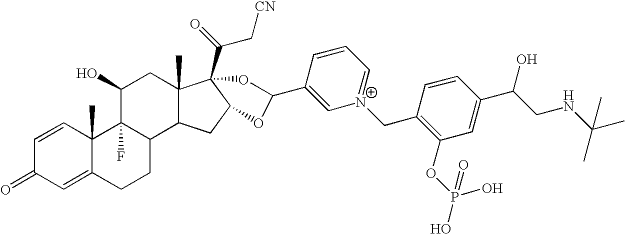

The title mutual prodrug can be prepared according to the procedure described in Example 106 starting from the pyridinium salt described in Example 138.

### Example 140

### Cytokine Release Inhibition

TABLE 1
General Procedures for the in vitro assays.

| | | Control | |
|---|---|---|---|
| Assay | Cell origin | compound | Reference |
| TNF-α secretion (h) | PBMC | dexamethasone | Schindler |
| (PBMC) | | | (1990) |
| IL-1β secretion (h) | PBMC | cycloheximide | Schindler |
| (PBMC) | | | (1990) |
| Cell viability (h) | PBMC | erythromycin | Mosmann |
| (PBMC/24 h) | | | (1983) |
| Immunosuppression | splenic lymphocytes | cyclosporin A | Soulillou |
| | isolated from C57BL/6 mice | | (1975) |
| | (5 × 10⁵ cells) and CBA mice | | |
| | (2.5 × 10⁵ cells) | | |

TABLE 2
Experimental conditions of the assays.

| | | | | Method of |
|---|---|---|---|---|
| Assay | Substrate/Stimulus/Tracer | Incubation | Reaction Product | Detection |
| TNF-α secretion (h) | LPS (1 μg/ml) | 24 h/37° C. | TNF-α | EIA |
| (PBMC) | | | | |
| IL-1β secretion (h) | LPS (1 μg/ml) | 24 h/37° C. | IL-1β | EIA |
| (PBMC) | | | | |
| Cell viability (h) | MTT (0.5 mg/ml) | 24 h./37° C. | formazan | Photometry |
| (PBMC/24 h) | | | | |
| Immunosuppression | Mouse splenic lymphocytes | 72 h./37° C. | [³H]TMD | Scintillation |
| | isolated from CBA mice | | incorporation | counting |
| | (2.5 × 10⁵ cells)/[³H]TMD | | | |
| | (1 μCi) | | | |

### Analysis and Expression of Results

The results are expressed as a percent of control values obtained in the presence of the test compounds. The IC₅₀ values (concentration causing a half-maximal inhibition of control values) were determined by non-linear regression analysis of the inhibition curves using Hill equation curve fitting.

TABLE 3
Cytokine release inhibition (IC₅₀ in nM).

| | TNF-α | IL-1 β | Immuno- |
|---|---|---|---|
| Compound Example | secretion | secretion | suppresion |
| 22 | 12 | 2.1 | 1.5 |
| 23 | 1.2 | — | 0.34 |
| 26 | Not active | >1000 | 31 |
| 27^{A} | Not active | 36 | 12 |
| 34 | >1000 | — | 20 |
| 56 | Not active | >1000 | Not active |
| 57 | Not active | >1000 | >1000 |
| 62^{B} | 11 | 350 | 1.9 |
| 22R-epimer | 12 | — | 0.89 |
| 22S-epimer | >1000 | — | 36 |
| 59 | 85 | — | 2 |
| 107^{C} | 810 | — | 80 |
| 133^{D} | >1000 | — | 180 |
| 45 | Not active | — | >1000 |
| 47 | Not active | — | 66 |
| 43 | >1000 | — | 8.8 |

(All compounds presented in the Table 3 were not cytotoxic (cell viability ca. 100%) up to 1000 nM).
^{A}steroid 13;
^{B}steroid 14;
^{C}mutual prodrug 16;
^{D}mutual prodrug 17.
“Not active” - IC₅₀ is not calculable because of less than 25% inhibition was observed at the highest tested concentration 1000 nM.

The selected compounds of the invention were tested in a panel of standard, cell-based in vitro assays evaluating the cytokine release inhibition and thus the anti-inflammatory activity of a test article. Several potent steroid analogs were identified, namely compounds described in Examples 23, 27, 43, 59 and 62. The mutual prodrugs of Examples 107 and 133 (compounds 16 and 17, respectively) have proven to be less active or inactive as compared to the steroid drugs (Examples 27 and 62, respectively). Therefore by masking the pharmacological properties of a respective steroid the mutual prodrug mitigates the oropharyngeal side effects and confines the antiinflammatory activity of a steroid to the endobronchial space, where the lung enzymes (specifically alkaline phosphatase) release the pharmacologically active steroid (see Examples 141-143).

### Example 141

### General Procedure for Conversion of the Mutual Steroid-β-Agonist Prodrugs to Salmeterol and Steroid after Exposure to Alkaline Phosphatase

Reaction and control solutions were prepared by adding a 500 μL aliquot of a ˜200 ng/μl solution in 1:1 acetonitrile/water of and the compound 16 (or alternatively 17) to 500 μl of a pH 7.4 buffer solution, containing 5 mM tris(hydroxymethyl)aminomethane, 1 mM ZnCl₂, 1 mM MgCl₂. For the reaction solutions, the buffer also contained approximately 600 ng/μl of alkaline phosphatase (Sigma-Aldrich) whereas the control buffer solutions contained no enzyme. The reaction and control solutions were incubated at 37° C. for 25 to 50 hours. The solutions were analyzed periodically for the respective mutual prodrug and reaction products by LCMS.

### Example 142

### Reaction of the Mutual Prodrug 16 with Alkaline Phosphatase to Yield Salmeterol and the Steroid 13

The mutual prodrug 16 (described in Example 107) was reacted with alkaline phosphatase according to the general procedure of Example 141, to produce salmeterol and the steroid 13 (described in Example 27). The concentration of the alkaline phosphatase in the reaction buffer was ˜600 ng/μL (the enzyme activity of the solution was not determined).

Only the mutual prodrug 16 was detected in the control solution (without enzyme). The reaction solution (with enzyme) showed the disappearance of the mutual prodrug 16, the initial appearance followed by the disappearance of the des-phosphorylated intermediate, and the appearance of salmeterol and the steroid compound 13 (as shown in Scheme VIII). Selected time points measured in this experiment are presented in Table 4. For the graphic representation of the enzymatic conversion see FIG. 1.

TABLE 4
Concentration of compounds detected in the ALP experiment.

| Hours | Prodrug 16 | Des-PO₄ | Steroid 13 | Salmeterol |
|---|---|---|---|---|
| @ | Concentration | Intermediate | Concentration | Concentration |
| 37° C. | nmol/ml | Peak Area | nmol/ml | nmol/ml |
| 0.00 | 91.0 | 3.78 × 10⁷ | 0.0 | 0.1 |
| 0.59 | 87.7 | 3.61 × 10⁸ | 1.6 | 1.8 |
| 1.19 | 78.6 | 4.78 × 10⁸ | 4.1 | 4.4 |
| 2.96 | 67.8 | 6.05 × 10⁸ | 15.3 | 12.3 |
| 3.56 | 62.3 | 6.09 × 10⁸ | 20.2 | 14.4 |
| 4.15 | 61.6 | 5.97 × 10⁸ | 21.6 | 17.1 |
| 10.67 | 43.1 | 4.03 × 10⁸ | 49.5 | 34.2 |
| 15.41 | 36.7 | 2.76 × 10⁸ | 54.6 | 41.4 |
| 19.56 | 33.1 | 2.02 × 10⁸ | 62.9 | 44.8 |
| 24.30 | 29.3 | 1.40 × 10⁸ | 67.3 | 46.9 |
| 30.82 | 24.8 | 9.51 × 10⁷ | 69.3 | 48.1 |
| 34.97 | 23.0 | 7.15 × 10⁷ | 66.5 | 49.6 |

### Example 143

### Reaction of the Mutual Prodrug 17 with Alkaline Phosphatase to Yield Salmeterol and the Steroid 14

The mutual prodrug 17 (described in Example 133) was reacted with alkaline phosphatase according to the general procedure of Example 141, to produce salmeterol and the steroid 14 (described in Example 62). The concentration of the alkaline phosphatase in the buffer added to the stock solution was ˜600 ng/μl (the enzyme activity of the solution was not determined).

Only the mutual prodrug 17 was detected in the control solution (without enzyme). The reaction solution (with enzyme) showed the disappearance of the mutual prodrug, the initial appearance followed by the disappearance of the des-phosphorylated intermediate, and the appearance of salmeterol and the steroid 14 (as shown in Scheme VIII). Selected time points measured in this experiment are presented in Table 5. For the graphic representation of the enzymatic conversion see FIG. 2.

TABLE 5
Concentration of compounds detected in the ALP experiment.

| Hours | Prodrug 17 | Des-PO₄ | Steroid 14 | Salmeterol |
|---|---|---|---|---|
| @ | Concentration | Intermediate | Concentration | Concentration |
| 37° C. | nmol/ml | Peak Area | nmol/ml | nmol/ml |
| 0.00 | 214.4 | 2.78 × 10⁷ | 0.0 | 0.0 |
| 0.53 | 112.6 | 4.03 × 10⁸ | 4.0 | 2.9 |
| 1.05 | 44.9 | 6.01 × 10⁸ | 9.9 | 8.9 |
| 2.10 | 9.8 | 6.60 × 10⁸ | 22.6 | 21.0 |
| 3.16 | 4.2 | 5.83 × 10⁸ | 34.8 | 31.1 |
| 4.21 | 3.7 | 5.74 × 10⁸ | 45.4 | 39.2 |
| 10.52 | 0.0 | 3.98 × 10⁸ | 88.1 | 80.1 |
| 19.99 | 0.0 | 2.48 × 10⁸ | 121.9 | 105.5 |
| 29.46 | 0.0 | 1.55 × 10⁸ | 137.6 | 120.6 |
| 39.99 | 0.0 | 9.68 × 10⁷ | 150.2 | 129.9 |
| 49.46 | 0.0 | 6.00 × 10⁷ | 169.2 | 135.3 |

1. A compound of the formula I or II 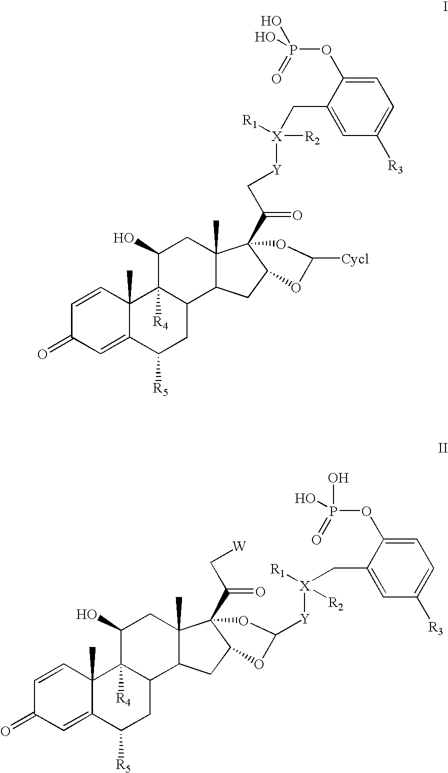
and pharmaceutical acceptable salts thereof, wherein:
X is S, N or a nitrogen-containing heterocycle in which the nitrogen atom in the heterocycle is linked to R₁ and R₂;
W is selected from the group consisting of Cl, F, OH, ONO₂, OCO-alkyl, OCO-aryl, CN, S-alkyl, and S-aryl;
Cycl is cycloalkyl or cycloalkyl with carbon atom(s) substituted with S or O;
Y is either absent or —Z(CH₂)n where n=0-6 and Z is S, O, N or N-alkyl;
R₁ and R₂ are independently selected from the group consisting of hydrogen, aryl, loweralkyl and substituted loweralkyl, or absent, or taken together to form a nonaromatic ring having 2-10 atoms selected from C, O, S, and N;
R₃ is 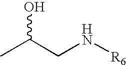
where R₆ is an alkyl group of 1-12 carbon atoms, arylalkyl or substituted arylalkyl with 1-3 CH₂ groups in the carbon chain substituted with atom(s) selected from O, S and N, and
R₄ and R₅ are independently H, Cl or F.

2. A compound of formula I as in claim 1 wherein: Cycl is cyclohexyl, R₁ is methyl, R₂ is absent, Y is N(CH₂)ₙ linked with X to form a piperazine ring,
R₃ is 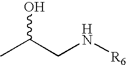
where R₆ is (CH₂)₆O(CH₂)₄Ph or tert-butyl, R₄ is F and R₅ is H.

3. A compound of formula I as in claim 1 wherein: Cycl is cyclohexyl, R₁ is methyl, R₂ is absent, Y is absent, X is S,
R₃ is 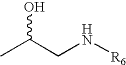
where R₆ is (CH₂)₆O(CH₂)₄Ph or tert-butyl, R₄ is F and R₅ is H.

4. A compound of formula II as in claim 1 wherein: Y, R₁ and R₂ are absent and X forms 4-tetrathiohydropyranyl ring, W is OH or CN
R₃ is 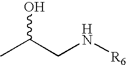
where R₆ is (CH₂)₆O(CH₂)₄Ph or tert-butyl, R₄ is F and R₅ is H.

5. A compound of formula II as in claim 1 wherein: Y, R₁ and R₂ are absent and X forms a 3-pyridyl ring, W is OH or CN
R₃ is 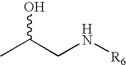
where R₆ is (CH₂)₆O(CH₂)₄Ph or tert-butyl, R₄ is F and R₅ is H.

6. The process of synthesis of compounds of claim 1.

7. A compound as in claim 1 selected from the group consisting of:
Salmeterol-phosphate-16,17-[(Cyclohexylmethylene)bis(oxy)]-9-fluoro-11-hydroxy-21-(4-methylpiperazinium)-pregna-1,4-diene-3,20-dione[11β,16α(R)];
Albuterol-phosphate-16,17-[(Cyclohexylmethylene)bis(oxy)]-9-fluoro-11-hydroxy-21-(4-methylpiperazinium)-pregna-1,4-diene-3,20-dione[11β,16α(R)];
Salmeterol-phosphate-16,17-[(Cyclohexylmethylene)bis(oxy)]-9-fluoro-11-hydroxy-21-methylsulfonium-pregna-1,4-diene-3,20-dione[11β,16α(R)];
Albuterol-phosphate-16,17-[(Cyclohexylmethylene)bis(oxy)]-9-fluoro-11-hydroxy-21-methylsulfonium-pregna-1,4-diene-3,20-dione[11β,16α(R)];
Salmeterol-phosphate-16,17-[(Tetrahydro-thiopyranylium)bis(oxy)]-9-fluoro-11,21-dihydroxy-pregna-1,4-diene-3,20-dione[11β,16α(R)];
Albuterol-phosphate-16,17-[(Tetrahydro-thiopyranylium)bis(oxy)]-9-fluoro-11,21-dihydroxy-pregna-1,4-diene-3,20-dione[11β,16α(R)];
Salmeterol-phosphate-16,17-[Pyridynium-3-methylene)bis(oxy)]-9-fluoro-11,21-dihydroxypregna-1,4-diene-3,20-dione[11β,16α];
Albuterol-phosphate-16,17-[Pyridynium-3-methylene)bis(oxy)]-9-fluoro-11,21-dihydroxypregna-1,4-diene-3,20-dione[11β,16α];
Salmeterol-phosphate-16,17-[Pyridynium-3-methylene)bis(oxy)]-9-fluoro-11-hydroxy-21-cyano-pregna-1,4-diene-3,20-dione[11β,16α]; and
Albuterol-phosphate-16,17-[Pyridynium-3-methylene)bis(oxy)]-9-fluoro-11-hydroxy-21-cyano-pregna-1,4-diene-3,20-dione[11β,16α].

8. A compound of the formula III: 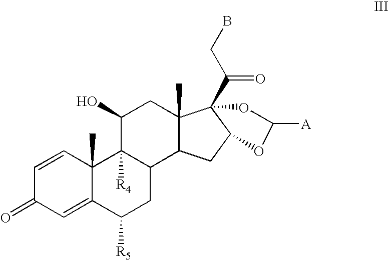
or pharmaceutically acceptable salts thereof, wherein:
A is cycloalkyl (with carbon atom(s) optionally substituted with S, O or NR₁), pyridyl or substituted pyridyl;
B is selected from the groups consisting of NR₁R₂, imidazolyl, CN, SCN, SR₁, Cl, F, OH, ONO₂, OCO-alkyl and OCO-aryl;
R₁ and R₂ are independently selected from the group consisting of hydrogen, aryl, heteroaryl, loweralkyl and substituted loweralkyl, or absent, or taken together to form a nonaromatic ring having 2-10 atoms selected from C, O, S, and N.

9. A compound as in claim 8 selected from the group consisting of:
16,17-[(Cyclohexylmethylene)bis(oxy)]-9-fluoro-11-hydroxy-21-(4-methylpiperazin-yl)-pregna-1,4-diene-3,20-dione[11β,16α(R)];
16,17-[(Cyclohexylmethylene)bis(oxy)]-9-fluoro-11-hydroxy-21-methylthio-pregna-1,4-diene-3,20-dione[11β,16α(R)];
16,17-[(Tetrahydro-thiopyran-4-yl)bis(oxy)]-9-fluoro-11,21-dihydroxy-pregna-1,4-diene-3,20-dione[11β,16α(R)];
16,17-[Pyridynyl-3-methylene)bis(oxy)]-9-fluoro-11,21-dihydroxypregna-1,4-diene-3,20-dione[11β,16α]; and
16,17-[Pyridynyl-3-methylene)bis(oxy)]-9-fluoro-11-hydroxy-21-cyano-pregna-1,4-diene-3,20-dione[11β,16α].

10. An aerosol formulation for the prevention and treatment of pulmonary inflammation and bronchoconstriction, said formulation comprising from about 10 μg to about 1000 μg of at least one substituted phenylphosphate mutual prodrug of claim 1 wherein said formulation is adapted to be administered by aerosolization to produce predominantly aerosol particles between 1 and 5μ.

11. An aerosol formulation as in claim 1 wherein the mutual prodrug is prepared as a dry powder and the formulation is administered using a dry powder inhaler.

12. An aerosol formulation for the prevention and treatment of pulmonary inflammation or bronchoconstriction, said formulation comprising from about 10 μg to about 1000 μg of at least one mutual prodrug of claim 1 wherein said formulation is adapted to be administered by aerosolization to produce predominantly aerosol particles between 1 and 5μ.

13. An aerosol formulation for the prevention and treatment of pulmonary inflammation or bronchoconstriction, said formulation comprising from about 10 μg to about 1000 μg of at least one mutual prodrug of claim 1 prepared as a dry powder for aerosol delivery in a physiologically compatible and tolerable matrix wherein said formulation is adapted to be administered using a dry powder inhaler able to produce predominantly aerosol particles between 1 and 5μ.

14. A method for the prevention and treatment of pulmonary inflammation or bronchoconstriction, comprising administering to a patient in need of such treatment an effective amount of an aerosol formulation comprising about 10 μg to about 1000 μg of at least one substituted phenylphosphate mutual prodrug as in claim 1.

15. A method as in claim 14 wherein when the mutual prodrug is delivered to the lung, the phosphate group is cleaved by an endogenous enzyme and the steroid and the β-agonist are individually released in a simultaneous manner.
